(19) United States
(12) Patent Application Publication
(10) Pub. No.: US 2012/0142723 A1
(43) Pub. Date: Jun. 7, 2012

(54) ACE2 ACTIVATOR COMPOUNDS AND METHODS OF USE THEREOF

(75) Inventors: David A. Ostrov, Gainesville, FL (US); Mohan K. Raizada, Alachua, FL (US); Jose A. Hernandez, Gainesville, FL (US)

(73) Assignee: University of Florida Research Foundation, Inc., Gainesville, FL (US)

(21) Appl. No.: 12/516,176

(22) PCT Filed: Nov. 21, 2007
(86) PCT No.: PCT/US07/24345

§ 371 Date: Jan. 28, 2010

Related U.S. Application Data
(60) Provisional application No. 60/860,894, filed on Nov. 22, 2006.

(51) Int. Cl. A61K 31/473; A61K 31/366; G06G 7/48; A61P 9/00; A61P 9/12; A61K 31/352; A61K 31/36

(52) U.S. Cl. 514/290; 514/455; 703/11

(57) ABSTRACT

The invention relates to methods of treating cardiovascular and cardiopulmonary diseases and associated conditions, including hypertension. The invention further relates to pharmaceutical compositions for treating cardiovascular and cardiopulmonary diseases, especially hypertension, and lung injury.

### CROSS-REFERENCE TO RELATED APPLICATIONS

This application claims the benefit of U.S. Provisional Patent Application No. 60/860,894, filed Nov. 22, 2006, the contents of which are incorporated herein by reference in their entirety.

### STATEMENT OF RIGHTS TO INVENTIONS MADE UNDER FEDERALLY SPONSORED RESEARCH

This work was supported by the National Institutes of Health, Grant Nos. NIH/HL56921 and NIH/HL33610. The government has certain rights in the invention.

### BACKGROUND OF THE INVENTION

ACE2 is a family member of the peptidylpeptidase angiotensin-converting enzymes (ACE), which are reviewed in Kem & Brown, N. Eng. J. Med. 323(16) 1136-1137 (1990), see also Yamada et al, Circ. Res. 68 141-149 (1991). There are three ACE enzymes currently known, ACE1, ACE2 and ACE3. (Cambien et al, Am. J. Hum. Genet. 43 774-780 (1988); Mattu et al. Circulation 91 270-274 (1995); Rigat et al, Nuc. Acids. Res. 20(6) 1433 (1992)). The human ACE gene (DCP1) is found on chromosome 17q23 and contains a restriction fragment length polymorphism consisting of the presence (Insertion, I) or absence (Deletion, D) of a 287 base pair alu repeat sequence in intron 16. ACE-2 (GenBank Accession No. AF291820) has been described by Donoghue, et al. (2000) Circ. Res. 87:e1-e9. ACE2 cleaves angiotensin I, but ACE-2 is a carboxypeptidase. The nucleic acid and amino acid sequences of ACE-2 reveal that certain portions of the ACE-2 protein and cDNA have a significant homology to certain regions of previously identified angiotensin converting enzymes (Altschul et al. J. Mol Biol. (1990)215:403).

The crystal structure of ACE2 was solved and revealed a “hinge” that is inhibitor-dependent and brings catalytic residues into position. Towler P, Staker Prasad S G, Menon S, Tang J, Parsons T, Ryan D, Fisher M, Williams D, Dales N A, Patane M A, and Pantoliano M W, ACE2 X-ray structures reveal a large hinge-bending motion important for inhibitor binding and catalysis, J Biol Chem. 2004, 23; 279(17):17996-8007.

Angiotensin-converting enzyme 2 (ACE2) is a type I membrane-anchored peptidyl carboxypeptidase of 805 amino acids (Donoghue et al. 2000, Tipnis et al. 2000). Its catalytic domain consists of approximately 733 residues and is 42% identical to that of its closest homolog, ACE. Unlike the ubiquitously expressed ACE, ACE2 is expressed only in the kidneys, heart (including all cardiovascular tissues), and lungs (Donoghue et al. 2000). Its substrate specificity has also been established to be different, and likely complementary, to that of ACE (Vickers et al. 2002). While ACE activity mainly results in the production of angiotensin II involved in vasoconstriction and the biosynthesis of aldosterone (an important regulator of blood pressure), ACE2 product peptides, namely angiotensin 1-7, are involved in vasodilation and hypotension. Furthermore, inhibitors of ACE such as captopril, lisinopril and enalaprilat do not significantly affect the activity of ACE2 (Donoghue et al. 2000, Tipnis et al., 2000).

Specific roles of ACE2 in different diseases and normal physiology are currently a subject of intense study. Nonetheless, its central role in the renin-angiotensin system (Burrel et al. 2004), cardiac contractile function (Crackower et al. 2002), hypertension (Katovich et al. 2005) and therefore cardiovascular disease have all been recently established. Crackower and others (2002) also observed an inverse correlation of ACE2 mRNA and blood pressure in experimental hypertension models. Other studies have begun to demonstrate ACE2 represents a tractable gene therapy target (Katovich et al. 2005; Huentelman et al. 2004). The approach attempts to over-express ACE2 to offer protection against cardiac hypertrophy and fibrosis (Katovich et al. 2005). The inhibition of ACE is an established therapeutic approach and presently one of the primary strategies for the treatment of hypertension. However these studies (mentioned above) clearly suggests that suppression of ACE and enhancement of ACE2 activity are both highly desirable to prevent and treat hypertension and related cardiovascular diseases.

Although ACE2 is homologous to ACE, the crystal structures of recombinant ACE2 (Towler et al. 2004) and testicular ACE (Natesh et al. 2003) clearly demonstrate structural differences. These differences are observed in the active site, helping rationalize their substrate specificity, and also in their general architecture. It is noted that no large conformational changes were observed between the free and inhibitor bound forms of ACE, while one of the largest hinge-bending motions was observed for ACE2. This may be a crystallization artifact, allowing ACE to only crystallize in the more compact conformation whether inhibitor is found or not.

### BRIEF SUMMARY OF THE INVENTION

In one aspect, the invention provides a method of treating a subject suffering from or susceptible to cardiovascular disease or cardiopulmonary disease or hypertension comprising administering to subject in need thereof a therapeutically effective amount of a compound capable of activating ACE2, or a pharmaceutically acceptable salt or prodrug thereof. In one embodiment, the compound is capable of binding to or interacting with a binding pocket defined (at least in part) by structure coordinates of one or more ACE2 amino acid residues Lys94, Tyr196, Gly205 and His 195. In another embodiment, the compound is capable of binding to or interacting with a binding pocket defined (at least in part) by structure coordinates of one or more ACE2 residues Gln98, Gln101 and Gly205. In certain embodiments, the compound is a compound disclosed herein, e.g., a compound of Formulae I or II, or one of compounds 3, 6 or 100-109, or a compound of Table 1, or a pharmaceutically acceptable ester, salt, or prodrug thereof.

In another aspect, the invention provides a method of treating a subject suffering from or susceptible to cardiovascular disease or cardiopulmonary disease or hypertension, comprising administering to the subject an effective amount of a compound capable of activating ACE2 activity or expression in a cell, such that the subject is treated.

In another aspect, the invention provides a method for identifying a compound that activates ACE2, the method comprising obtaining a crystal structure of ACE2 or obtaining information relating to the crystal structure of ACE2, and modeling a test compound into or on the crystal structure coordinates to determine whether the compound activates ACE2. In certain embodiments, the step of modeling comprises modeling or determining the ability of the compound to bind to or associate with a binding pocket defined by structure coordinates of one or more ACE2 amino acid residues Lys94, Tyr196, Gly205 and His195. In another embodiment, the step of modeling comprises modeling or determining the ability of the compound to bind to or associate with a binding pocket defined by structure coordinates of one or more ACE2 amino acid residues Gln98, Gln101 and Gly205.

Yet another aspect of the invention is a method for identifying a compound that modulates the activity of ACE2, the method comprising using the atomic coordinates of one or more ACE2 amino acid residues Lys94, Tyr196, Gly205 and His195 to generate a three-dimensional structure of a molecule comprising an ACE2 binding pocket, and employing the three-dimensional structure to identify a compound that modulates (e.g., activates the activity of ACE2.

In another aspect, the invention provides a method of treating a subject suffering from or susceptible to acute lung injury, comprising administering to the subject an effective amount of an ACE2 activator compound, such that the subject is treated.

In another aspect, the invention provides a method of treating a subject suffering from or susceptible to acute lung injury, comprising administering to the subject an effective amount of a compound capable of activating ACE2 activity or expression in a cell, such that the subject is treated.

In another aspect, the invention provides a method of treating a subject suffering from or susceptible to pulmonary hypertension, comprising administering to the subject an effective amount of an ACE2 activator compound, such that the subject is treated.

In another aspect, the invention provides a method of treating a subject suffering from or susceptible to pulmonary hypertension, comprising administering to the subject an effective amount of a compound capable of activating ACE2 activity or expression in a cell, such that the subject is treated.

In another aspect, the invention provides a method of treating a subject suffering from or susceptible to cardiac or renal fibrosis, the method comprising administering to a subject in need thereof a therapeutically effective amount of an ACE2 activator compound, such that the subject is treated.

In another aspect, the invention provides a method of treating a subject suffering from or susceptible to cardiac or renal fibrosis, the method comprising administering to a subject in need thereof a therapeutically effective amount of a compound capable of activating ACE2 activity or expression in a cell, such that the subject is treated.

Yet another aspect of the invention is a method for identifying a compound that modulates the activity of ACE2, the method comprising using the atomic coordinates of one or more ACE2 amino acid residues Gln98, Gln101 and Gly205 to generate a three-dimensional structure of a molecule comprising an ACE2 binding pocket, and employing the three-dimensional structure to identify a compound that modulates (e.g., activates the activity of ACE2.

In another aspect, the invention provides a method for increasing activity or expression of ACE2 in a cell or a subject, the method comprising contacting the cell or subject with an effective amount of a compound capable of increasing activity or expression of ACE2, such that activity or expression of ACE2 is increased.

In another aspect, the invention provides a packaged composition including a therapeutically effective amount of an ACE2 activator compound and a pharmaceutically acceptable carrier or diluent. The composition may be formulated for treating a subject suffering from or susceptible to cardiovascular disease or an associated condition (such as stroke or heart disease), or hypertension, and packaged with instructions to treat a subject suffering from or susceptible to cardiovascular disease or an associated condition (such as stroke or heart disease), or hypertension.

In one aspect, the invention provides a kit for treating cardiovascular disease or an associated condition (such as stroke or heart disease), or hypertension, or pulmonary hypertension or acute lung injury, in a subject is provided and includes a compound disclosed herein, e.g., a compound of Formulae I or II, or one of compounds 3, 6 or 100-109, or a compound of Table 1, or a pharmaceutically acceptable ester, salt, or prodrug thereof, and instructions for use. In further aspects, the invention provides kits for treating cardiovascular disease or an associated condition (such as stroke or heart disease), or hypertension, assessing the efficacy of an anti-cardiovascular disease (or hypertension) treatment in a subject using an ACE2 activator, monitoring the progress of a subject being treated with an ACE2 activator, selecting a subject with or susceptible to cardiovascular disease or an associated condition (such as stroke or heart disease), or hypertension, and/or treating a subject suffering from or susceptible to cardiovascular disease or an associated condition (such as stroke or heart disease), or hypertension. In certain embodiments, the invention provides: a kit for treating cardiovascular disease or an associated condition (such as stroke or heart disease), or hypertension, in a subject, the kit comprising a compound capable of increasing activity (or expression) of ACE2, or pharmaceutically acceptable esters, salts, and prodrugs thereof, and instructions for use; in certain embodiments, the Compound is represented by any of the structures of Formulae I or II, or one of compounds 3, 6 or 100-109, or a compound of Table 1, or a pharmaceutically acceptable salt thereof; in certain embodiments, the compound is selected from the group consisting of Compound 3 ((1-[(2-(diethylamino)ethyl]amino]-4-(hydroxymethyl)-7-[[(4-methylphenyl)sulfonyl]oxy]-9H-xanthen-9-one)) and Compound 6 (resorcinalnaphthalein).

In another aspect, the invention relates to a three-dimensional structure of ACE2. The invention provides the key structural features of ACE2, particularly the shape of small-molecule binding pockets remote from the active site of ACE2.

Thus, the present invention provides molecules or molecular complexes that comprise one or more of binding pockets (e.g., Pocket 1, as described herein) or homologues of a binding pocket that have similar three-dimensional shapes.

The invention also provides a pharmaceutical composition of the compounds described herein, e.g., a compound of Formulae I or II, or one of compounds 3, 6 or 100-109, or a compound of Table 1, or a pharmaceutically acceptable ester, salt, and prodrug thereof. The pharmaceutical composition comprises a compound described herein, or a pharmaceutically acceptable ester, salt, or prodrug thereof, together with a pharmaceutically acceptable carrier.

In another aspect, the invention provides a machine readable storage medium which comprises the structural coordinates of a binding pocket defined (at least in part) by structure coordinates of one or more of ACE2 amino acid residues Gln98, Gln101 and Gly205, or a homologous binding pocket.

In another aspect, the invention provides a machine readable storage medium which comprises the structural coordinates of a binding pocket defined (at least in part) by structure coordinates of one or more of ACE2 amino acid residues Lys94, Tyr196, Gly205 and His195, or a homologous binding pocket.

In another aspect, the invention provides a computer for producing a three-dimensional representation of a molecule or molecular complex, wherein said molecule or molecular complex comprises a binding pocket defined by structural coordinates of a binding pocket defined (at least in part) by structure coordinates of one or more of ACE2 amino acid residues Gln98, Gln101 and Gly205, or a homologous binding pocket; or b) a three-dimensional representation of a homologue of said molecule or molecular complex, wherein said homologue comprises a binding pocket that has a root mean square deviation from the backbone atoms of said amino acids of not more than about 2.0 angstroms. The computer includes (i) a machine-readable data storage medium comprising a data storage material encoded with machine-readable data, wherein said data comprises the structural coordinates of a binding pocket defined (at least in part) by structure coordinates of one or more of ACE2 amino acid residues Gln98, Gln101 and Gly205, or a homologous binding pocket; (ii) a working memory for storing instructions for processing said machine-readable data; (iii) a central-processing unit coupled to said working memory and to said machine-readable data storage medium for processing said machine readable data into said three-dimensional representation; and (iv) a display coupled to said central-processing unit for displaying said three-dimensional representation.

In another aspect, the invention provides a computer for producing a three-dimensional representation of a molecule or molecular complex, wherein said molecule or molecular complex comprises a binding pocket defined by structural coordinates of a binding pocket defined (at least in part) by structure coordinates of one or more of ACE2 amino acid residues Lys94, Tyr196, Gly205 and His195, or a homologous binding pocket; or b) a three-dimensional representation of a homologue of said molecule or molecular complex, wherein said homologue comprises a binding pocket that has a root mean square deviation from the backbone atoms of said amino acids of not more than about 2.0 angstroms. The computer includes (i) a machine-readable data storage medium comprising a data storage material encoded with machine-readable data, wherein said data comprises the structural coordinates of a binding pocket defined (at least in part) by structure coordinates of one or more of ACE2 amino acid residues Lys94, Tyr196, Gly205 and His195, or a homologous binding pocket; (ii) a working memory for storing instructions for processing said machine-readable data; (iii) a central-processing unit coupled to said working memory and to said machine-readable data storage medium for processing said machine readable data into said three-dimensional representation; and (iv) a display coupled to said central-processing unit for displaying said three-dimensional representation.

The invention also provides methods for designing, evaluating and identifying compounds which bind to the aforementioned binding pockets. Such compounds are potential activators or enhancers of ACE2 activity. Other embodiments of the invention are disclosed infra.

In another aspect, the invention provides a packaged composition comprising a therapeutically effective amount of an angiotensin converting enzyme (ACE2) activator and a pharmaceutically acceptable carrier or diluent is presented. The composition may be formulated for treating a subject suffering from or susceptible to cardiovascular disease or an associated condition, or hypertension or pulmonary hypertension, and packaged with instructions to treat a subject suffering from or susceptible to cardiovascular disease or an associated condition, or hypertension or pulmonary hypertension.

In one aspect, the invention provides a kit for treating cardiovascular disease or an associated condition, or hypertension or pulmonary hypertension in a subject. The kit comprises a compound of Table 1, or a pharmaceutically acceptable ester, salt, or prodrug thereof, and instructions for use. In further aspects, kits for treating or preventing cardiovascular disease, assessing the efficacy of an anti-cardiovascular-disease treatment in a subject, monitoring the progress of a subject being treated with an ACE activator, selecting a subject suffering from or susceptible to cardiovascular disease or an associated condition, or hypertension or pulmonary hypertension, for treatment with an ACE activator, and/or treating a subject suffering from or susceptible to cardiovascular disease or an associated condition, or hypertension are provided.

In any of the aspects of the invention, the compound can be, e.g., a compound of Formulae I or II, or one of compounds 3, 6 or 100-109, or a compound of Table 1, or a pharmaceutically acceptable ester, salt, or prodrug thereof.

In another aspect, the invention provides a compound represented by Formula (II):

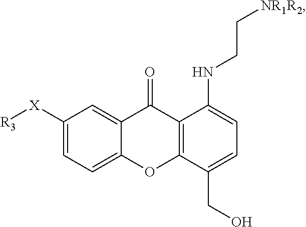

in which X is O or S; R₁ and R₂ are independently hydrogen, optionally substituted C₁-C₈alkyl, optionally substituted C₃-C₈cycloalkyl, optionally substituted C₂-C₈ alkenyl, optionally substituted C₂-C₈alkynyl, optionally-substituted C₁-C₈alkanoyl, or optionally substituted aryl; and R₃ is optionally substituted C₁-C₈alkyl, optionally substituted C₃-C₈cycloalkyl, optionally substituted C₂-C₈ alkenyl, optionally substituted C₂-C₈alkynyl, optionally substituted C₁-C₈alkanoyl, optionally substituted C₁-C₈alkanoyl or optionally substituted C₁-C₈alkylsulfonyl, optionally substituted C₁-C₈arylsulfonyl, or optionally substituted aryl; or a pharmaceutically acceptable salt or prodrug thereof.

In certain embodiments of Formula (II), R₁ and R₂ are each methyl. In certain embodiments of Formula (II), X is O. In certain embodiments of Formula (II), R₃ is optionally substituted C₁-C₈alkanoyl. In certain embodiments of Formula (II), R₃ is optionally substituted C₁-C₈arylsulfonyl. In certain embodiments of Formula (II), the compound is not 1-[[2-(diethylamino)ethyl]amino]-4-(hydroxymethyl)-7-[[(4-methylphenyl)sulfonyl]oxy]-9H-xanthen-9-one.

In another aspect, the invention provides a pharmaceutical composition comprising a compound of Formula II and a pharmaceutically acceptable carrier.

Other aspects and embodiments of the invention are disclosed infra.

### BRIEF DESCRIPTION OF THE DRAWINGS

The present invention is further described below with reference to the following non-limiting examples and with reference to the following figures, in which:

FIG. 1. (a) Free (open) and inhibitor bound (closed) ACE2 structures (PDBID: 1R42 and 1R4L respectively) used in structural analysis to identify differences in the molecular surface of the two conformations. (b) Sphere clusters targeting three sites on ACE2. (b) Shows the structure of the inhibitor bound conformation of ACE2 (inhibitor not shown). The cluster for site 1 was generated based on the structure of the open form of the enzyme but it is shown superposed on the closed form to show its relative position to the other clusters. The view of the structure is rotated 90° around the horizontal axis. (c) and (e) Molecular docking models of ACE2 activators XNT and resorcinolnaphthalein, respectively. Compounds were minimized and treated as flexible ligands during molecular docking calculations. Searching parameters were made increasingly more thorough until the docking scores converged. (c) and (e) show ACE2 in a similar orientation. Likely hydrogen bonding interactions are labeled with dashed lines. (d) and (f) chemical structures of XNT and resorcinolnaphthalein, respectively. (g) ACE2-specific enhancement by XNT and resorcinolnaphthalein (100 μM). ACE2 activators have no effect on ACE activity in the same conditions. * p<0.001.

FIG. 2. Compound 3 from site 1 activates ACE2. Concentrations ranging from 0-800 μM clearly gave a clean dose response even though the compound did not go completely into solution. Assays done in 10 nM enzyme, 10 μM substrate, 100 mM NaCl, 75 mM tris pH7.5 and 0.5 μM ZnCl₂ at room temperature. The 30 minute time course yielded linear curves (A) from where rates were calculated (B). All curves in the top panel had a straight line correlation coefficient of >0.98, except 20 μM compound (c.c.=0.93).

FIG. 3. Compound 6 from site 1 activates ACE2. (A) shows the activity of ACE2 is significantly increased by about 2-fold. Assay done in 100 μM Compound 6. Error bars are standard errors of measurement at a 95% confidence interval. The curves show a 40 minute time course obtained in identical conditions to those described in FIG. 2. (B) shows rates in RFU/s from control (in triplicate: C+1, C+2, C+3) and compound concentrations ranging from 0-500 μM. 20, 50, and 100 μM gave identical curves and were pooled to obtain the average shown in the top panel.

FIG. 4. The ACE2 activator compounds do not enhance ACE activity. Top panel shows activation of ACE2 by compound 3 at 50 μM. Error bars are standard errors of measurement at 95% confidence intervals. Bottom panel shows the activity of ACE (red) is not enhanced by either Compound 3 (dark blue, 100 μM; dark purple, 50 μM) or Compound 6 (bright blue, 100 μM; magenta, 50 μM). All assays were done in triplicate but in panel (B) error bars are omitted for simplicity.

FIG. 5. Acute infusion of an ACE2 activator compound decreases mean arterial pressure (MAP) in SHR rats.

FIG. 6. Chronic infusion of an ACE2 activator compounds decreases mean arterial pressure (MAP) and heart rate (HR) in SHR rats.

FIG. 7. Arterial blood pressure was measured directly in awake freely moving rats as described in Methods. XNT administration induced a dose-dependent decrease in BP of (a) WKY rats and (b) SHR. However, the effect in SHR was more significant. These effects were accompanied by a significant decrease in the HR of (c) WKY rats and (d) SHR. *p<0.05, **p<0.01 and ***p<0.001 compared with vehicle injection, n=3-9.

FIG. 8. Functional effects of chronic infusion of XNT. Nine rats in each group were fitted with osmotic minipumps and infused with vehicle (black bullets) or XNT at 60 μg/day (white bullets). Indirect BP was monitored as described in Methods. (a) Effect of chronic XNT infusion in BP of SHR and WKY rats. The decrease of BP started at the first week of infusion and it achieved the maximal decrease by the third week in SHR (p<0.05 n=9). (b, c) Effect of BK on BP in WKY (b) and SHR (c). After 28 days of XNT infusion, as described previously, rats were injected with the indicated doses of BK and BP was monitored as described in Methods. The BK effect was more pronounced in hypertensive rats. XNT treatment potentiated the BK hypotensive effect in both strains. (d, e) Cardiac function in isolated hearts from XNT-treated SHR. Chronic infusion of XNT resulted in an increase (n=8) in (d)+dP/dt and (e)−dP/dt in the SHR. *p<0.05 and *** p<0.001 compared with vehicle-infused rats (n=6).

FIG. 9. Effect of XNT on cardiac and renal fibrosis. After termination of chronic infusion protocols, the hearts and kidneys were dissected out, sectioned and stained with Sirius red as described in Methods. Myocardial, perivascular and renal interstitial fibrosis were examined and quantified as described in Methods. Significant increase in myocardial (b) and perivascular (e) fibrosis was observed in SHR compared with WKY rats (a and d, respectively). Significant reduction in myocardial (c) and perivascular (f) fibrosis was observed in XNT-treated SHR. In the SHR kidney there was a significant increase in interstitial fibrosis (j) compared to the WKY rat (i). This was also diminished in XNT-treated SHR (k). (g, h, i) Collagen deposit quantification as described in the methods. *p<0.05 compared to SHR, n=2-8.

FIG. 10. Effect of XNT on Ang-(1-7) immunoreactivity in hearts and cardiac fibroblasts. Animals from chronic experiments were sacrificed, hearts removed, sectioned and used for immunohistochemical analyses as described in Methods. Endogenous Ang-(1-7) immunoreactivity was found in cardiomyocytes (white asterisks) and in (a) interstitial and (c) perivascular fibroblasts (white arrows) of SHR. XNT-treated hearts demonstrate significantly more Ang-(1-7) immunoreactive fibroblasts (b, d). Cultured cardiac fibroblasts treated with vehicle showed little Ang-(1-7) immunoreactivity (f). A significant increase in the intensity Ang-(1-7) immunoreactivity was seen when cultures were treated with 100 μM XNT for 1 hour (g). Negative controls were obtained by omission of the primary antibody from the incubation procedure (e). Black asterisks: vascular wall.

FIG. 11. Effect of XNT on ACE2 immunoreactivity in hearts and cardiac fibroblasts. The experimental protocol was essentially the same as for FIG. 5. Little ACE2 immunoreactivity was found in cardiomyocytes (white asterisks) and in (a) interstitial and (c) perivascular fibroblasts (white arrows) in vehicle-treated SHR. However, chronic infusion of XNT resulted in increases in the numbers and intensity of ACE2 positive cardiac fibroblasts, but not in cardiomyocytes (b, d). This was confirmed with the use of cardiac fibroblasts in culture. Endogenous ACE2 activity was observed in vehicle-treated fibroblasts (f) but XNT treatment caused a significant increase in ACE2 immunostaining (g). Negative controls were obtained by omission of the primary antibody from the incubation procedure (e). Black asterisks: vascular wall.

### DETAILED DESCRIPTION OF THE INVENTION

### 1. Definitions

Before further description of the present invention, and in order that the invention may be more readily understood, certain terms are first defined and collected here for convenience.

As used herein, the term “acute lung injury” refers to conditions generally involving bilateral pulmonary infiltrates on chest X-ray, a pulmonary capillary wedge pressure of less than 18 mm Hg, and a PaO₂/FiO₂ of less than 300. Acute lung injury includes hypoxemic respiratory syndrome and acute respiratory distress syndrome (ARDS). ARDS is one of the most severe forms of acute lung injury. ARDS is a serious clinical syndrome with a high mortality rate (30-60%). ARDS may be caused by include sepsis, pulmonary aspiration, pneumonias, major trauma, burns, and infections (e.g., with the severe acute respiratory syndrome (SARS) coronavirus).

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral; inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally effect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and sentences is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and still more preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “biological activities” of a compound of the invention includes all activities elicited by compound of the inventions in a responsive cell or subject. It includes genomic and non-genomic activities elicited by these compounds.

“Biological composition” or “biological sample” refers to a composition containing or derived from cells or biopolymers. Cell-containing compositions include, for example, mammalian blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or, other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In a preferred embodiment, a suitable biological composition or biological sample is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes red blood cells and/or platelets and/or leukocytes and/or bone marrow cells.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., sufficient to treat cardiovascular disease or an associated condition. An effective amount of compound of the invention may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

A therapeutically effective amount of compound of the invention (i.e., an effective dosage) may range from about 0.001 to 30 mg/kg body weight, or from about 0.01 to 10 mg/kg body weight, or from about 0.05 to 5 mg/kg body weight, or from about 0.1 to 1 mg/kg, 0.2 to 0.9 mg/kg, 0.3 to 0.8 mg/kg, 0.4 to 0.7 mg/kg, or 0.5 to 0.6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, preferably, can include a series of treatments. In one example, a subject is treated with a compound of the invention in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound of the invention used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “haloalkyl” is intended to include alkyl groups as defined above that are mono-, or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “improved biological properties” refers to any activity inherent in a compound of the invention that enhances its effectiveness in vivo. In a preferred embodiment, this term refers to any qualitative or quantitative improved therapeutic property of a compound of the invention, such as reduced toxicity.

The term “optionally substituted” is intended to encompass groups that are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₃-C₈cycloalkyl, C₂-C₈ alkenyl, C₂-C₈alkynyl, C₁-C₈alkoxy, C₂-C₈alkyl ether, C₃-C₈alkanone, C₁-C₈alkylthio, amino, mono- or di-(C₁-C₈alkyl)amino, haloC₁-C₈alkyl, C₁-C₈alkoxy, C₁-C₈alkanoyl, C₂-C₈alkanoyloxy, C₁-C₈alkoxycarbonyl, —COON, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substitutents).

The term “isomers” or “stereoisomers” refers to compounds which have identical chemical constitution, but differ with regard to the arrangement of the atoms or groups in space.

The term “obtaining” as in “obtaining the ACE activator” is intended to include purchasing, synthesizing or otherwise acquiring the ACE activator.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to an amount of a compound of the invention of the formula (I) or otherwise described herein which is effective, upon single or multiple dose administration to the patient, in preventing or treating cardiovascular disease or cardiopulmonary disease or hypertension or cardiac or renal fibrosis.

The language “reduced toxicity” is intended to include a reduction in any undesired side effect elicited by a compound of the invention when administered in vivo.

The term “sulfhydryl” or “thiol” means —SH.

The term “subject” includes organisms which are capable of suffering from cardiovascular disease, or an associated condition (including hypertension) or who could otherwise benefit from the administration of a compound of the invention of the invention, such as human and non-human animals. Preferred human animals include human patients suffering from or prone to suffering from cardiovascular disease or associated state, including hypertension, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc. “Susceptible to a cardiovascular disease or associated state, including hypertension” is meant to include subjects at risk of developing cardiovascular disease or associated state, including hypertension, i.e., subjects suffering from existing cardiovascular disease or associated state, including hypertension, subjects having risk factors (such as overweight) for cardiovascular disease or associated state, including hypertension, etc.

The phrases “systemic administration,” “administered systemically”, “peripheral administration” and “administered peripherally” as used herein mean the administration of a compound of the invention(s), drug or other material, such that it enters the patient's system and, thus, is subject to metabolism and other like processes, for example, subcutaneous administration.

The language “therapeutically effective amount” of a compound of the invention of the invention refers to an amount of an agent which is effective, upon single or multiple dose administration to the patient, in treating or preventing cardiovascular disease or an associated condition or symptom, including hypertension, or in prolonging the survivability of the patient with such condition beyond that expected in the absence of such treatment.

The language “cardiovascular disease or associated condition” refers to a condition of the heart or vasculature, including heart disease and stroke, which can be prevented, treated or otherwise ameliorated by administration of one or more compounds of the invention (e.g., is caused, exacerbated or characterized by insufficient ACE2 activity). Other examples of cardiovascular disease or associated conditions include cardiac hypertrophy and fibrosis.

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

### 2. Compounds of the Invention

In one aspect, the invention provides a compound capable of activating ACE2 activity. In certain embodiments, the compound is capable of activating or increasing ACE2 activity selectively, e.g., without concomitant activation of ACE activity. In certain embodiments, the ACE2 activator compound can be represented by the Formula (I):

Ar—(Y)ₙ (I)

wherein,

Ar is a polycyclic fused aromatic moiety;

Y represents a hydrogen bond donor or acceptor; and

n is an integer from 2 to 8; or a pharmaceutically acceptable salt or prodrug thereof.

In certain embodiments, Ar is a polycyclic moiety having at least two, three, four, five, or six fused rings, including spirocyclic rings. In certain embodiments, each hydrogen bond donor or acceptor is independently selected from the group consisting of —OH, O-alkyl, O-aryl; NH₂, NH-alkyl, NH-aryl; N(alkyl)(aryl), N(alkyl)₂; N(aryl)₂; COOH; COO-alkyl; or a salt thereof. In certain embodiments, Ar may be substituted with one or more groups selected from: alkyl (e.g., lower alkyl), alkenyl, alkynyl, alkylaryl, aryl (including heteroaryl), halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, and alkylaryl.

In certain embodiments, the compound is represented by Formula (II):

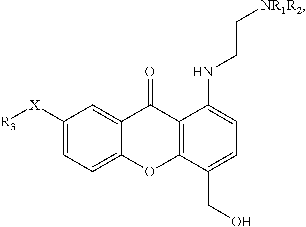

in which X is O or S; R₁ and R₂ are independently hydrogen, optionally substituted C₁-C₈alkyl, optionally substituted C₃-C₈cycloalkyl, optionally substituted C₂-C₈ alkenyl, optionally substituted C₂-C₈alkynyl, optionally substituted C₁-C₈alkanoyl, or optionally substituted aryl; and R₃ is optionally substituted C₁:C₈alkyl, optionally substituted C₃-C₈cycloalkyl, optionally substituted C₂-C₈ alkenyl, optionally substituted C₂-C₈alkynyl, optionally substituted C₁-C₈alkanoyl, optionally substituted C₁-C₈alkanoyl or optionally substituted C₁-C₈alkylsulfonyl, optionally substituted C₁-C₈arylsulfonyl, or optionally substituted aryl; or a pharmaceutically acceptable salt or prodrug thereof.

In certain embodiments of Formula (II), R₁ and R₂ are each methyl. In certain embodiments of Formula (II), X is O. In certain embodiments of Formula (II), R₃ is optionally substituted C₁-C₈alkanoyl. In certain embodiments of Formula (II), R₃ is optionally substituted C₁-C₈arylsulfonyl. In certain embodiments of Formula (II), the compound is not 1-[[2-(diethylamino)ethyl]amino]-4-(hydroxymethyl)-7-[[(4-methylphenyl)sulfonyl]oxy]-9H-xanthen-9-one.

In certain embodiments, the compound is

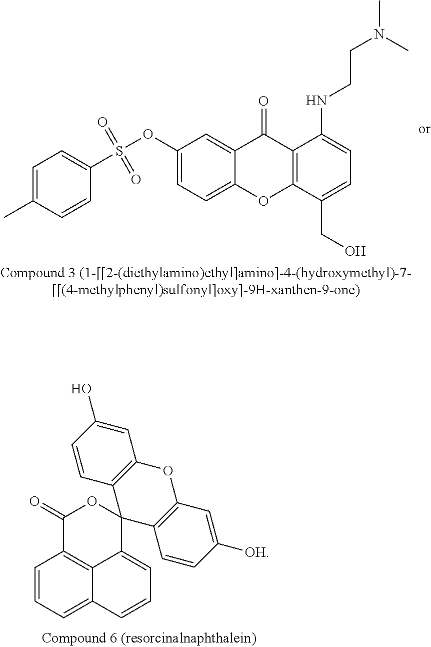

In certain embodiments, a compound of the invention can be represented by any of the following structures:

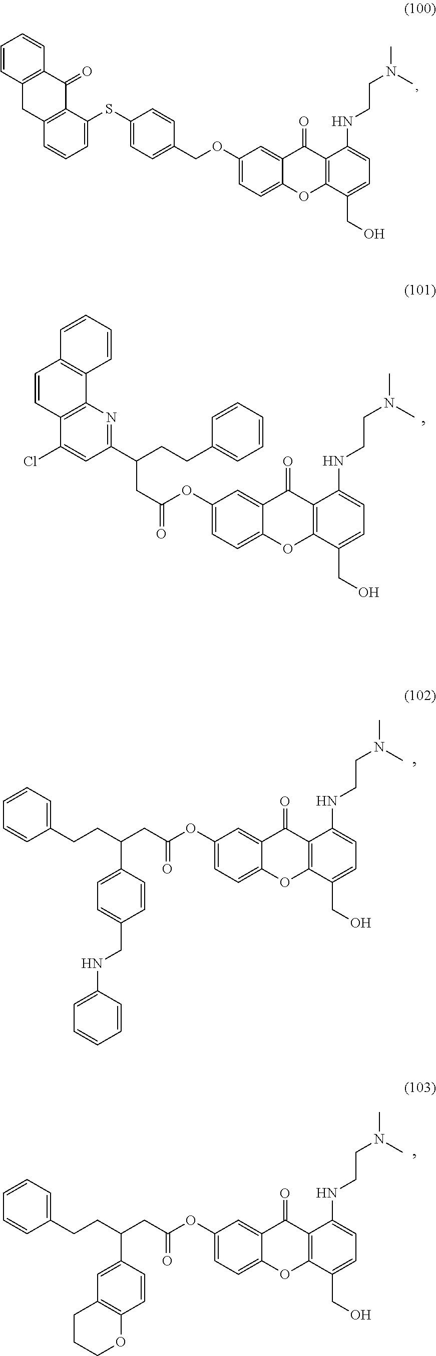

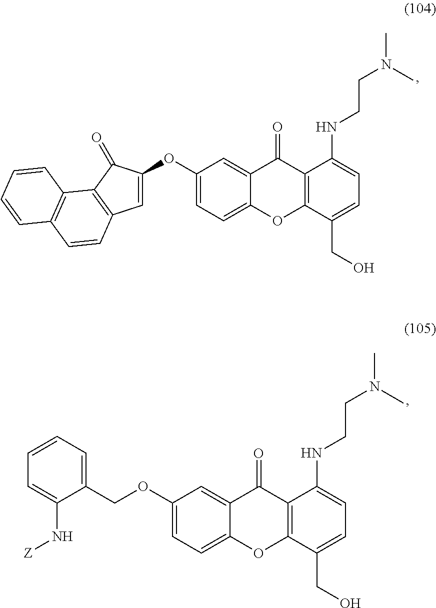

wherein Z is a bridged polycycle (for example, a group of the structure:

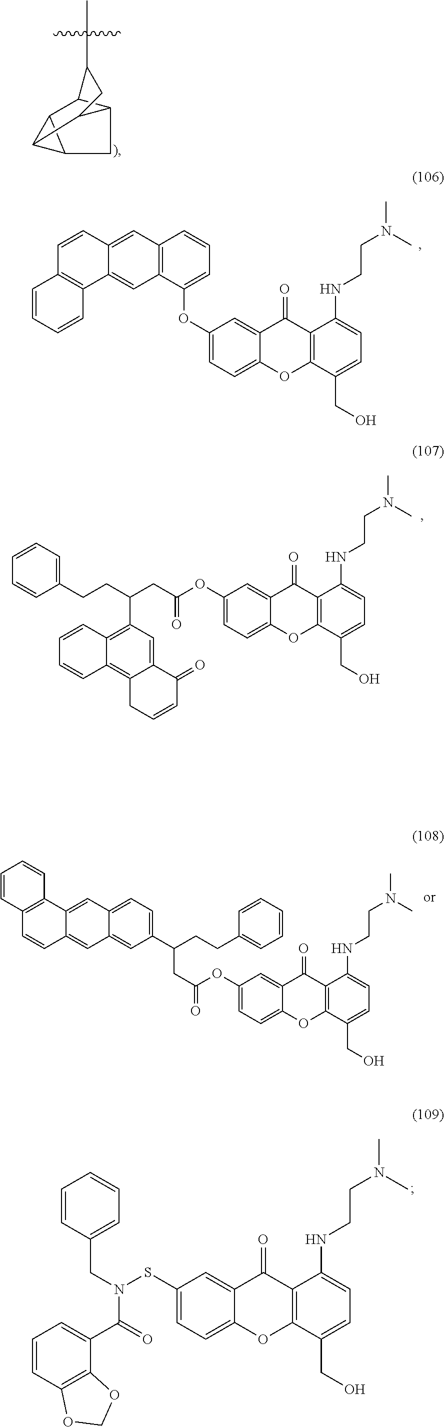

or a pharmaceutically acceptable salt or prodrug thereof.

In general, a compound of the invention will be selected such that the compound is capable of binding to a binding pocket of ACE2 that is defined (at least in part) by structure coordinates of one or more of ACE2 amino acid residues Lys94, Tyr196, Gly205 and His195, or is capable of binding to a binding pocket of ACE2 that is defined (at least in part) by structure coordinates of one or more of ACE2 amino acid residues Gln98, Gln101 and Gly205. Moreover, in certain embodiments, a compound has one or more of the following properties: (1) not more than 5 hydrogen bond donors; (2) not more than 10 hydrogen bond acceptors; (3) a molecular weight of 1000 or less, 800 or less, 600 or less, 500 or less; and (4) a partition coefficient log P of less than 5.

Compounds according to the invention can generally be made according to techniques known in the art (see, e.g., Comprehensive Organic Synthesis, Trost, B. M. and Fleming, I. eds., Pergamon Press, Oxford; and references cited therein). Furthermore, compounds of the invention can be purified, separated, or isolated, e.g., by crystallization, chromatographic separation (e.g., by liquid chromatography), or by other methods known in the art.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Uses of the Compounds of the Invention

As described herein below, it has now surprisingly been found that the compounds of the invention and analogs can treat and prevent cardiovascular diseases, including systemic hypertension or pulmonary hypertension. Thus, in one embodiment, the invention provides a method of treating a subject suffering from or susceptible to cardiovascular disease or systemic or pulmonary hypertension comprising administering to subject in need thereof a therapeutically effective amount of a compound capable of activating ACE2, or a pharmaceutically acceptable salt or prodrug thereof. In one embodiment, the compound is capable of binding to or interacting with a binding pocket defined (at least in part) by structure coordinates of one or more ACE2 amino acid residues Lys94, Tyr196, Gly205 and His195. In another embodiment, the compound is capable of binding to or interacting with a binding pocket defined (at least in part) by structure coordinates of one or more ACE2 residues Gln98, Gln101 and Gly205. In certain embodiments, the compound is a compound disclosed herein, e.g., a compound of Formula I or II, or one of compounds 100-109, or a compound of Table 1. In certain embodiments, the subject is a mammal, e.g., a primate, e.g., a human.

In another aspect, the invention provides a method of treating a subject suffering from or susceptible to cardiovascular disease or hypertension, comprising administering to the subject an effective amount of a compound capable of activating ACE2 activity or expression in a cell, such that the subject is treated.

In one aspect, the invention provides a method of treating a subject suffering from or susceptible to cardiovascular disease or hypertension comprising administering to subject in need thereof a therapeutically effective amount of a compound capable of activating ACE2, or a pharmaceutically acceptable salt or prodrug thereof. In one embodiment, the compound is capable of binding to or interacting with a binding pocket defined (at least in part) by structure coordinates of one or more ACE2 amino acid residues Lys94, Tyr196, Gly205 and His195. In another embodiment, the compound is capable of binding to or interacting with a binding pocket defined (at least in part) by structure coordinates of one or more ACE2 residues Gln98, Gln101 and Gly205. In certain embodiments, the compound is a compound disclosed herein, e.g., a compound of Table 1.

In another aspect, the invention provides a method of treating a subject suffering from or susceptible to cardiovascular disease or hypertension, comprising administering to the subject an effective amount of a compound capable of activating ACE2 activity or expression in a cell, such that the subject is treated.

In one aspect, the invention provides a method of treating a subject suffering from or susceptible to pulmonary hypertension comprising administering to subject in need thereof a therapeutically effective amount of a compound capable of activating ACE2, or a pharmaceutically acceptable salt or prodrug thereof. In one embodiment, the compound is capable of binding to or interacting with a binding pocket defined (at least in part) by structure coordinates of one or more ACE2 amino acid residues Lys94, Tyr196, Gly205 and His195. In another embodiment, the compound is capable of binding to or interacting with a binding pocket defined (at least in part) by structure coordinates of one or more ACE2 residues Gln98, Gln101 and Gly205. In certain embodiments, the compound is a compound disclosed herein, e.g., a compound of Table 1.

In another aspect, the invention provides a method of treating a subject suffering from or susceptible to acute lung injury, comprising administering to the subject an effective amount of an ACE2 activator compound, such that the subject is treated.

In another aspect, the invention provides a method of treating a subject suffering from or susceptible to acute lung injury, comprising administering to the subject an effective amount of a compound capable of activating ACE2 activity or expression in a cell, such that the subject is treated.

In another aspect, the invention provides a method of treating a subject suffering from or susceptible to cardiac or renal fibrosis, the method comprising administering to a subject in need thereof a therapeutically effective amount of an ACE2 activator compound, such that the subject is treated. In certain embodiments, a method of treating a subject suffering from cardiac or renal fibrosis includes ameliorating, decreasing the extent of, or reversing cardiac or renal fibrosis in an organ or a subject.

In another aspect, the invention provides a method of treating a subject suffering from or susceptible to cardiac or renal fibrosis, the method comprising administering to a subject in need thereof a therapeutically effective amount of a compound capable of activating ACE2 activity or expression in a cell, such that the subject is treated. In certain embodiments, a method of treating a subject suffering from cardiac or renal fibrosis includes ameliorating, decreasing the extent of, or reversing cardiac or renal fibrosis in an organ or a subject.

In another aspect, the invention provides a method for increasing activity or expression of ACE2 in vitro, or in a cell or a subject, the method comprising contacting the cell or subject with an effective amount of a compound capable of increasing activity or expression of ACE2, such that activity or expression of ACE2 is increased.

In certain embodiments, the methods of the invention include administering to a subject a therapeutically effective amount of a compound of the invention in combination with another pharmaceutically active compound. Examples of pharmaceutically active compounds include compounds known to treat cardiovascular disease or hypertension, such as ACE inhibitors, angiotension II receptor blockers, diuretics, beta blockers, calcium channel blockers, statins, aspirin, and the like. Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference. The compound of the invention and the pharmaceutically active compound may be administered to the subject in the same pharmaceutical composition or in different pharmaceutical compositions (at the same time or at different times).

Determination of a therapeutically effective amount or a prophylactically effective amount of the compound of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective amount or dose, and the prophylactically effective amount or dose, a number of factors are considered by the attending clinician, including, but not limited to: the specific cardiovascular disease or condition involved; pharmacodynamic characteristics of the particular agent and its mode and route of administration; the desired time course of treatment; the species of mammal; its size, age, and general health; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective amount of a compound of the invention of the invention is expected to vary from about 0.1 milligram per kilogram of body weight per day (mg/kg/day) to about 100 mg/kg/day.

Compounds determined to be effective for the prevention or treatment of cardiovascular disease in animals, e.g., dogs, chickens, and rodents, may also be useful in treatment of similar conditions in humans. Those skilled in the art of treatment in humans will know, based upon the data obtained in animal studies, the dosage and route of administration of the compound to humans. In general, the dosage and route of administration in humans is expected to be similar to that in animals.

The identification of those patients who are in need of prophylactic treatment for cardiovascular disease states is well within the ability and knowledge of one skilled in the art. Certain of the methods for identification of patients which are at risk of developing cardiovascular disease states which can be treated by the subject methods are appreciated in the medical arts, such as family history, the presence of other risk factors associated with the development of that disease state in the subject patient, and the like. A clinician skilled in the art can readily identify such candidate patients, by the use of, for example, clinical tests, physical examination and medical/family/travel history.

A method of assessing the efficacy of an anti-cardiovascular disease treatment in a subject includes determining the physical condition of the subject (e.g., blood pressure, degree or extent of atherosclerosis, and the like) and then administering a therapeutically effective amount of an ACE activator compound of the invention to the subject. After a appropriate period of time after the administration of the compound, e.g., 2 hours, 4 hours, 8 hours, 12 hours, or 72 hours, or one week, the physical condition of the subject is determined again. The modulation of the cardiovascular disease state indicates efficacy of an treatment. The physical condition of the subject may be determined periodically throughout treatment. For example, the physical condition of the subject may be checked every few hours, days or weeks to assess the further efficacy of the treatment. The method described may be used to screen or select patients that may benefit from treatment with an ACE activator.

As used herein, “obtaining a biological sample from a subject,” includes obtaining a sample for use in the methods described herein. A biological sample is described above.

In another aspect, the invention provides a method for identifying a compound that activates ACE2, the method comprising obtaining a crystal structure of ACE2 or obtaining information relating to the crystal structure of ACE2, and modeling a test compound into or on the crystal structure coordinates to determine whether the compound activates ACE2. In certain embodiments, the step of modeling comprises modeling or determining the ability of the compound to bind to or associate with a binding pocket defined by structure coordinates of one or more ACE2 amino acid residues Lys94, Tyr196, Gly205 and His195. In another embodiment, the step of modeling comprises modeling or determining the ability of the compound to bind to or associate with a binding pocket defined by structure coordinates of one or more ACE2 amino acid residues Gln98, Gln101 and Gly205.

Yet another aspect of the invention is a method for identifying a compound that modulates the activity of ACE2, the method comprising using the atomic coordinates of one or more ACE2 amino acid residues Lys94, Tyr196, Gly205 and His 195 to generate a three-dimensional structure of a molecule comprising an ACE2 binding pocket, and employing the three-dimensional structure to identify a compound that modulates (e.g., activates the activity of ACE2.

Yet another aspect of the invention is a method for identifying a compound that modulates the activity of ACE2, the method comprising using the atomic coordinates of one or more ACE2 amino acid residues Gln98, Gln101 and Gly205 to generate a three-dimensional structure of a molecule comprising an ACE2 binding pocket, and employing the three-dimensional structure to identify a compound that modulates (e.g., activates the activity of ACE2.

In another aspect, a compound of the invention is packaged in a therapeutically effective amount with a pharmaceutically acceptable carrier or diluent. The composition may be formulated for treating a subject suffering from or susceptible to a cardiovascular disease or associated condition, and packaged with instructions to treat a subject suffering from or susceptible to such a disease or condition.

In another aspect, the invention provides a method for increasing activity or expression of ACE2 in a cell or a subject, the method comprising contacting the cell or subject with an effective amount of a compound capable of for increasing activity or expression of ACE2, such that activity or expression of ACE2 is increased.

In another aspect, the invention provides a packaged composition including a therapeutically effective amount of an ACE2 activator compound and a pharmaceutically acceptable carrier or diluent. The composition may be formulated for treating a subject suffering from or susceptible to cardiovascular disease or an associated condition (such as stroke or heart disease), or hypertension, and packaged with instructions to treat a subject suffering from or susceptible to cardiovascular disease or an associated condition (such as stroke or heart disease), or hypertension.

In one aspect, the invention provides a kit for treating cardiovascular disease or an associated condition (such as stroke or heart disease), or hypertension, in a subject is provided and includes a compound disclosed herein, e.g., a compound of Table 1, or a pharmaceutically acceptable ester, salt, and prodrug thereof, and instructions for use. In further aspects, the invention provides kits for treating cardiovascular disease or an associated condition (such as stroke or heart disease), or hypertension, assessing the efficacy of an anti-cardiovascular disease (or hypertension) treatment in a subject using an ACE2 activator, monitoring the progress of a subject being treated with an ACE2 activator, selecting a subject with or susceptible to cardiovascular disease or an associated condition (such as stroke or heart disease), or hypertension, or acute lung injury, and/or treating a subject suffering from or susceptible to cardiovascular disease or an associated condition (such as stroke or heart disease), or hypertension. In certain embodiments, the invention provides: a kit for treating cardiovascular disease or an associated condition (such as stroke or heart disease), or hypertension, in a subject, the kit comprising a compound capable of increasing activity (or expression) of ACE2, or pharmaceutically acceptable esters, salts, and prodrugs thereof, and instructions for use; in certain embodiments, the compound is represented by Formula I or II, or one of Compounds 100-109, or by any of the structures of Table 1, or a pharmaceutically acceptable salt thereof; in certain embodiments, the compound is selected from the group consisting of Compound 3 and Compound 6 (toluene-4-sulfonic acid 8-(2-dimethylamino-ethylamino)-5-hydroxymethyl-9-oxo-9H-xanthen-2-yl ester).

In another aspect, the invention provides the use of a compound of the invention for the manufacture of a medicament for the treatment of cardiovascular disease or cardiopulmonary disease (including systemic or pulmonary hypertension) or cardiac or renal fibrosis.

The present methods can be performed on cells in culture, e.g. in vitro or ex vivo, or on cells present in an animal subject, e.g., in vivo. Compounds of the inventions can be initially tested in vitro using primary cultures of cells.

The present methods can be performed on cells in culture, e.g. in vitro or ex vivo, or on cells present in an animal subject, e.g., in vivo. Compound of the invention can be initially tested in vitro using cells from the respiratory tract from embryonic rodent pups (See e.g. U.S. Pat. No. 5,179,109-fetal rat tissue culture), or other mammalian (See e.g. U.S. Pat. No. 5,089,517-fetal mouse tissue culture) or non-mammalian animal models.

Alternatively, the effects of a compound of the invention can be characterized in vivo using animals models.

### 4. Pharmaceutical Compositions

The invention also provides a pharmaceutical composition, comprising an effective amount of a compound of the invention of formula I or II, or Compounds 100-109, or Compounds 3 or 6, or a compound of Table 1, or otherwise described herein and a pharmaceutically acceptable carrier. In a further embodiment, the effective amount is effective to treat cardiovascular or cardiopulmonary disease or an associated condition, including hypertension, or cardiac or renal fibrosis, as described previously.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compound of the inventions of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the invention(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, more preferably from about 10 percent to about 30 percent.

Methods of preparing these compositions include the step of bringing into association a compound of the invention(s) with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the invention(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the invention(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound of the invention(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound of the invention(s) include powders, sprays, ointments, pastes, creams, lotions, gels, solutions, patches and inhalants. The active compound of the invention(s) may be mixed under sterile conditions with a pharmaceutically-acceptable carrier, and with any preservatives, buffers, or propellants which may be required.

The ointments, pastes, creams and gels may contain, in addition to compound of the invention(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention(s), excipients such as lactose, talc, silicic acid, aluminum hydroxide, calcium silicates and polyimide powder, or mixtures of these substances. Sprays can additionally contain customary propellants, such as chlorofluorohydrocarbons and volatile unsubstituted hydrocarbons, such as butane and propane.

The compound of the invention(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more compound of the invention(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound of the invention(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the invention(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound of the invention(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.01 to 10 mg per day.

A preferred dose of the compound of the invention for the present invention is the maximum that a patient can tolerate and not develop serious or unacceptable side effects. In certain embodiments, the compound of the present invention is administered at a concentration of about 10 micrograms to about 100 mg per kilogram of body weight per day, about 0.1-about 10 mg/kg or about 1.0 mg-about 10 mg/kg of body weight per day. Ranges intermediate to the above-recited values are also intended to be part of the invention.

### 5. Screening Methods and Systems

In another aspect, the invention provides a method for identifying a compound that activates ACE2, the method comprising obtaining a crystal structure of ACE2 or obtaining information relating to the crystal structure of ACE2, and modeling a test compound into or on the crystal structure coordinates to determine whether the compound activates ACE2. In certain embodiments, the step of modeling comprises modeling or determining the ability of the compound to bind to or associate with a binding pocket defined by structure coordinates of one or more ACE2 amino acid residues Lys94, Tyr196, Gly205 and His195. In another embodiment, the step of modeling comprises modeling or determining the ability of the compound to bind to or associate with a binding pocket defined by structure coordinates of one or more ACE2 amino acid residues Gln98, Gln101 and Gly205.

Yet another aspect of the invention is a method for identifying a compound that modulates the activity of ACE2, the method comprising using the atomic coordinates of one or more ACE2 amino acid residues Lys94, Tyr196, Gly205 and His 195 to generate a three-dimensional structure of a molecule comprising an ACE2 binding pocket, and employing the three-dimensional structure to identify a compound that modulates (e.g., activates the activity of ACE2.

Yet another aspect of the invention is a method for identifying a compound that modulates the activity of ACE2, the method comprising using the atomic coordinates of one or more ACE2 amino acid residues Gln98, Gln101 and Gly205 to generate a three-dimensional structure of a molecule comprising an ACE2 binding pocket, and employing the three-dimensional structure to identify a compound that modulates (e.g., activates the activity of ACE2.

In another aspect, the invention relates to a three-dimensional structure of ACE2. The invention provides the key structural features of ACE2, particularly the shape of small-molecule binding pockets remote from the active site of ACE2.

In another aspect, the invention relates to a method of identifying a modulator (e.g., an activator or enhancer of activity) for an enzyme (e.g., ACE2), the method comprising identifying a surface site on the enzyme, remote from the enzyme active site, and testing to determine whether a candidate compound binds to the remote site and modulates enzyme activity.

In another aspect, the invention provides a machine readable storage medium which comprises the structural coordinates of either one or both of the binding pockets identified herein, or similarly shaped, homologous binding pockets. Such storage medium encoded with these data are capable of displaying a three-dimensional graphical representation of a molecule or molecular complex which comprises such binding pockets on a computer screen or similar viewing device.

Thus, in one embodiment, invention provides a machine readable storage medium which comprises the structural coordinates of a binding pocket defined (at least in part) by structure coordinates of one or more of ACE2 amino acid residues Gln98, Gln101 and Gly205, or a homologous binding pocket.

In another embodiment, the invention provides a machine readable storage medium which comprises the structural coordinates of a binding pocket defined (at least in part) by structure coordinates of one or more of ACE2 amino acid residues Lys94, Tyr196, Gly205 and His195, or a homologous binding pocket.

In another aspect, the invention provides a computer for producing a three-dimensional representation of a molecule or molecular complex, wherein said molecule or molecular complex comprises a binding pocket defined by structural coordinates of a binding pocket defined (at least in part) by structure coordinates of one or more of ACE2 amino acid residues Gln98, Gln101 and Gly205, or a homologous binding pocket; or b) a three-dimensional representation of a homologue of said molecule or molecular complex, wherein said homologue comprises a binding pocket that has a root mean square deviation from the backbone atoms of said amino acids of not more than about 2.0 angstroms. The computer includes (i) a machine-readable data storage medium comprising a data storage material encoded with machine-readable data, wherein said data comprises the structural coordinates of a binding pocket defined (at least in part) by structure coordinates of one or more of ACE2 amino acid residues Gln98, Gln101 and Gly205, or a homologous binding pocket; (ii) a working memory for storing instructions for processing said machine-readable data; (iii) a central-processing unit coupled to said working memory and to said machine-readable data storage medium for processing said machine readable data into said three-dimensional representation; and (iv) a display coupled to said central-processing unit for displaying said three-dimensional representation.

In another aspect, the invention provides a computer for producing a three-dimensional representation of a molecule or molecular complex, wherein said molecule or molecular complex comprises a binding pocket defined by structural coordinates of a binding pocket defined (at least in part) by structure coordinates of one or more of ACE2 amino acid residues Lys94, Tyr196, Gly205 and His195, or a homologous binding pocket; or b) a three-dimensional representation of a homologue of said molecule or molecular complex, wherein said homologue comprises a binding pocket that has a root mean square deviation from the backbone atoms of said amino acids of not more than about 2.0 angstroms. The computer includes (i) a machine-readable data storage medium comprising a data storage material encoded with machine-readable data, wherein said data comprises the structural coordinates of a binding pocket defined (at least in part) by structure coordinates of one or more of ACE2 amino acid residues Lys94, Tyr196, Gly205 and His195, or a homologous binding pocket; (ii) a working memory for storing instructions for processing said machine-readable data; (iii) a central-processing unit coupled to said working memory and to said machine-readable data storage medium for processing said machine readable data into said three-dimensional representation; and (iv) a display coupled to said central-processing unit for displaying said three-dimensional representation.

Thus, the computer produces a three-dimensional graphical structure of a molecule or a molecular complex which comprises a binding pocket.

In another embodiment, the invention provides a computer for producing a three-dimensional representation of a molecule or molecular complex defined by structure coordinates of all or some of the ACE2 amino acids, or a three-dimensional representation of a homologue of said molecule or molecular complex, wherein said homologue comprises a binding pocket that has a root mean square deviation from the backbone atoms of said amino acids of not more than 2.0 (more preferably not more than 1.5) angstroms

In exemplary embodiments, the computer or computer system can include components which are conventional in the art, e.g., as disclosed in U.S. Pat. No. 5,978,740 and/or 6,183,121 (incorporated herein by reference). For example, a computer system can includes a computer comprising a central processing unit (“CPU”), a working memory (which may be, e.g., RAM (random-access memory) or “core” memory), a mass storage memory (such as one or more disk drives or CD-ROM drives), one or more cathode-ray tube (CRT) or liquid crystal display (LCD) display terminals, one or more keyboards, one or more input lines, and one or more output lines, all of which are interconnected by a conventional system bus.

Machine-readable data of this invention may be inputted to the computer via the use of a modem or modems connected by a data line. Alternatively or additionally, the input hardware may include CD-ROM drives, disk drives or flash memory. In conjunction with a display terminal, a keyboard may also be used as an input device.

Output hardware coupled to the computer by output lines may similarly be implemented by conventional devices. By way of example, output hardware may include a CRT or LCD display terminal for displaying a graphical representation of a binding pocket of this invention using a program such as QUANTA or PYMOL. Output hardware might also include a printer, or a disk drive to store system output for later use.

In operation, the CPU coordinates the use of the various input and output devices, coordinates data accesses from the mass storage and accesses to and from working memory, and determines the sequence of data processing steps. A number of programs may be used to process the machine-readable data of this invention, including commercially-available software.

A magnetic storage medium for storing machine-readable data according to the invention can be conventional. A magnetic data storage medium can be encoded with a machine-readable data that can be carried out by a system such as the computer system described above. The medium can be a conventional floppy diskette or hard disk, having a suitable substrate which may be conventional, and a suitable coating, which may also be conventional, on one or both sides, containing magnetic domains whose polarity or orientation can be altered magnetically. The medium may also have an opening for receiving the spindle of a disk drive or other data storage device.

The magnetic domains of the medium are polarized or oriented so as to encode in manner which may be conventional, machine readable data such as that described herein, for execution by a system such as the computer system described herein.

An optically-readable data storage medium also can be encoded with machine-readable data, or a set of instructions, which can be carried out by a computer system. The medium can be a conventional compact disk read only memory (CD-ROM) or a rewritable medium such as a magneto-optical disk which is optically readable and magneto-optically writable.

In the case of CD-ROM, as is well known, a disk coating is reflective and is impressed with a plurality of pits to encode the machine-readable data. The arrangement of pits is read by reflecting laser light off the surface of the coating. A protective coating, which preferably is substantially transparent, is provided on top of the reflective coating.

In the case of a magneto-optical disk, as is well known, a data-recording coating has no pits, but has a plurality of magnetic domains whose polarity or orientation can be changed magnetically when heated above a certain temperature, as by a laser. The orientation of the domains can be read by measuring the polarization of laser light reflected from the coating. The arrangement of the domains encodes the data as described above.

Structure data, when used in conjunction with a computer programmed with software to translate those coordinates into the 3-dimensional structure of a molecule or molecular complex comprising a binding pocket may be used for a variety of purposes, such as drug discovery.

For example, the structure encoded by the data may be computationally evaluated for its ability to associate with chemical entities. Chemical entities that associate with a binding pocket of ACE2 s disclosed herein may increase or activate ACE2 activity, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention relates to a method for evaluating the potential of a chemical entity to associate with a) a molecule or molecular complex comprising a binding pocket defined, at least in part, by structure coordinates of one or more ACE2 amino acid residues selected from Lys94, Tyr196, Gly205 and His195, as described herein, or b) a homologue of said molecule or molecular complex, wherein said homologue comprises a binding pocket that has a root mean square deviation from the backbone atoms of said amino acids of not more than 2.0 (more preferably 1.5) angstroms.

This method comprises the steps of:

i) employing computational means to perform a fitting operation between the chemical entity and a binding pocket of the molecule or molecular complex; and

ii) analyzing the results of the fitting operation to quantify the association between the chemical entity and the binding pocket. This embodiment relates to evaluating the potential of a chemical entity to associate with or bind to a binding pocket referred to herein as “Pocket #1”.

The term “chemical entity”, as used herein, refers to chemical compounds, complexes of at least two chemical compounds, and fragments of such compounds or complexes.

In an alternate embodiment, the same steps indicated above are used in a method for evaluating the potential of a chemical entity to associate with or bind to.

a) a molecule or molecular complex comprising a binding pocket defined, at least in part, by structure coordinates of one or more ACE2 amino acid residues selected from Gln98, Gln101 and Gly205, as described herein, or b) a homologue of said molecule or molecular complex, wherein said homologue comprises a binding pocket that has a root mean square deviation from the backbone atoms of said amino acids of not more than 2.0 (more preferably not more than 1.5) angstroms.

In certain embodiments, the method evaluates the potential of a chemical entity to associate with a molecule or molecular complex defined by structure coordinates of all or some of the amino acids of ACE2, as described herein, or a homologue of said molecule or molecular complex having a root mean square deviation from the backbone atoms of said amino acids of not more than 2.0 (more preferably not more than 1.5) angstroms.

In a further embodiment, the structural coordinates one of the binding pockets described herein can be utilized in a method for identifying a potential agonist or antagonist of a molecule comprising an ACE2 binding pocket. This method comprises the steps of

a) using the atomic coordinates of ACE2 amino acid residues Gln98, Gln101 and Gly205, as described herein, with a root mean square deviation from the backbone atoms of said amino acids of not more than about 2.0 (more preferably not more than 1.5) angstroms, to generate a three-dimensional structure of molecule comprising an ACE2 binding pocket;

b) employing the three-dimensional structure to design or select the potential agonist or antagonist. The method further includes the optional steps of c) synthesizing the agonist or antagonist; and d) contacting the agonist or antagonist with the molecule to determine the ability of the potential agonist or antagonist to interact with the molecule.

Alternatively, the atomic coordinates of the ACE2 amino acid residues Lys94, Tyr 196, Gly205 and His 195, may be used in step a), above, to generate a three-dimensional structure of molecule comprising an ACE2 binding pocket.

The present inventors' elucidation of heretofore unknown binding pockets in the structure of ACE2 provides the necessary information for designing new chemical entities and compounds that may interact with ACE2, in whole or in part, and may therefore modulate (e.g., increase) the activity of ACE2, preferably with selectivity relative to other ACEs.

The design of compounds that bind to ACE2 binding pockets according to this invention generally involves consideration of several factors. First, the entity must be capable of physically and structurally associating with parts or all of the ACE2 binding pockets. Non-covalent molecular interactions important in this association include hydrogen bonding, van der Waals interactions, hydrophobic interactions and electrostatic interactions. Second, the entity must be able to assume a conformation that allows it to associate with the ACE2 binding pocket(s) directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the binding pocket or homologues thereof.

The potential inhibitory or binding effect of a chemical entity on a ACE2 binding pocket may be analyzed prior to its actual synthesis and testing by the use of computer modeling techniques. If the theoretical structure of the given entity suggests insufficient interaction and association between it and the target binding pocket, testing of the entity is obviated. However, if computer modeling indicates a strong interaction, the molecule may then be synthesized and tested for its ability to bind to a binding pocket. This may be achieved, e.g., by testing the ability of the molecule to activate ACE2 activity, e.g., using assays described herein or known in the art. In this manner, synthesis of inoperative compounds may be avoided.

A potential inhibitor of an ACE2-related binding pocket may be computationally evaluated by means of a series of steps in which chemical entities or fragments are screened and selected for their ability to associate with the ACE2-related binding pockets.

One skilled in the art may use one of several methods to screen chemical entities or fragments for their ability to associate with an ACE2 binding pocket. This process may begin by visual inspection of, for example, an ACE2 binding pocket on the computer screen based on the structure coordinates described herein, or other coordinates which define a similar shape generated from the machine-readable storage medium. Selected fragments or chemical entities may then be positioned in a variety of orientations, or docked, within that binding pocket as defined supra. Docking may be accomplished using software such as Quanta and DOCK, followed by energy minimization and molecular dynamics with standard molecular mechanics force fields, such as CHARMM and AMBER.

Specialized computer programs (e.g., as known in the art and/or commercially available and/or as described herein) may also assist in the process of selecting fragments or chemical entities:

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target binding pocket.

Instead of proceeding to build a compound capable of binding to a binding pocket in a step-wise fashion one fragment or chemical entity at a time as described above, inhibitory or other binding compounds may be designed as a whole or “de novo” using either an empty binding site or optionally including some portion(s) of a known inhibitor(s). There are many de novo ligand design methods known in the art, some of which are commercially available (e.g., LeapFrog, available from Tripos Associates, St. Louis, Mo.).

Other molecular modeling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)).

Once a compound has been designed or selected, the efficiency with which that entity may bind to a binding pocket may be tested and optimized by computational evaluation.

Specific computer software is available in the art to evaluate compound deformation energy and electrostatic interactions. Examples of programs designed for such uses include: AMBER; QUANTA/CHARMM (Accelrys, Inc., Madison, Wis.) and the like. These programs may be implemented, for instance, using a commercially-available graphics workstation. Other hardware systems and software packages will be known to those skilled in the art.

Another technique involves the in silico screening of virtual libraries of compounds, e.g., as described herein (see, e.g., the Examples hereinbelow). Many thousands of compounds can be rapidly screened and the best virtual compounds can be selected for further screening (e.g., by synthesis and in vitro testing). Small molecule databases can be screened for chemical entities or compounds that can bind, in whole or in part, to an ACE2 binding pocket. In this screening, the quality of fit of such entities to the binding site may be judged either by shape complementarity or by estimated interaction energy.

Finally, additional computational techniques can be used for automated structure-based optimization with software packages such as RACHEL (Tripos, Inc.). RACHEL allows a database of fragments to be screened and evaluated (i.e., scored) as each fragment is considered as an extension of the lead compound. The lead compound can then be grown in silico at user defined sites and ranked again. This approach can provide a “filtered” library of derivatives likely to have an increased affinity for the target.

The invention also provides methods for designing, evaluating and identifying compounds which bind to the aforementioned binding pockets. Such compounds are potential activators or enhancers of ACE2 activity. Other embodiments of the invention are disclosed herein.

The invention is further illustrated by the following examples which should in no way should be construed as being further limiting.

### EXAMPLES

### Materials and Methods

### Virtual Screening

The software package of DOCKv5.2 (Ewing et al. 2001) was used for in silico screening of ˜140,000 compounds available from the National Cancer Institute, Developmental Therapeutics Program. The structure coordinates and chemical information for each compound were processed either with accessory software from DOCK or with the ZINC server (Irwin and Shoichet 2005). Each compound was docked as a rigid body in 100 different orientations and before scoring the orientations were filtered by bump filter parameters, excluding compounds with extreme steric clashes.

The grid-based scoring system was used for scoring with the non-bonded force field energy function implemented in DOCK. A standard 6-12 Lennard-Jones potential was used to evaluate van der Waals contacts. Spheres were generated by SPHGEN (Kuntz et al. 1982) and clusters were edited by hand to target specific sites on the molecular surface of ACE2.

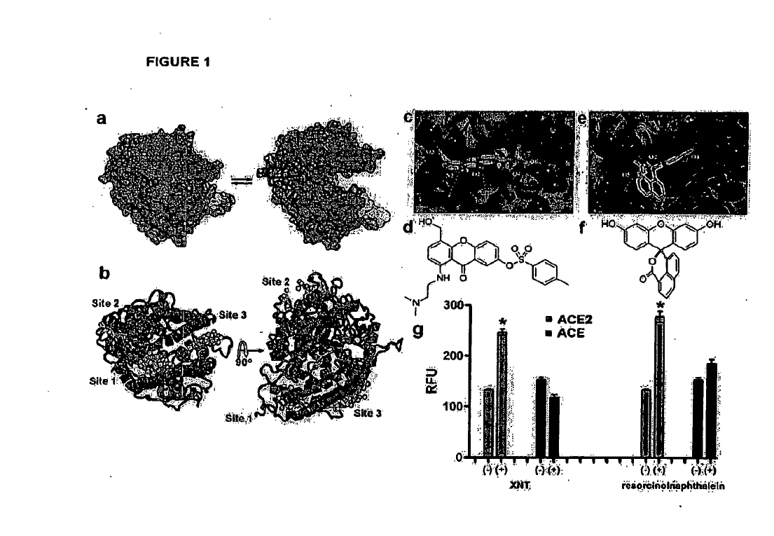

Three different molecular surface pockets, remote to the active site of ACE2, were targeted with spheres to rank the compounds of the NCI database (FIG. 1). Two sites were identified in the inhibitor bound form of the enzyme (sites 2 and 3), and a single site (site 1) was identified in the open conformation of ACE2. Each site was selected based on its uniqueness to each conformation. Thus, according to the crystal structures of ACE2 available from the Protein Data Bank (PDBID: 1R42 and 14RL, free and bound enzyme respectively) the structural pockets represented by sites 2 and 3 are not present in the open conformation of the enzyme. (The PDB file for PDBID: 1R42 is attached hereto as an Appendix which is incorporated herein in its entirety.) Likewise, site 1 seems to fill with amino acid side chains in the closed conformation. Molecular surfaces were visualized with the software GRASP (Nicholls et al. 1991) to show the concavity of surface pockets. Some pockets were more pronounced in one conformation or the other. Changes in the solvent accessible surface areas for each residue between the open and the closed conformations were also analyzed. Solvent accessible surface area changes were not as helpful in this case but may be used in the future to identify pockets by looking at residues that are exposed in one conformation but not the other.

After ranking with DOCK, the top scoring compounds for each site were tested in vitro with human recombinant ACE2. The top ten scoring compounds for each site were selected for functional testing. Active compounds were submitted to a more rigorous calculation with DOCK. Both compounds were docked in at least 3,000 orientations, energy minimized, and with flexible bond parameters on. Other parameter such as number of minimization steps and number of conformation steps were also increased to perform a more exhaustive search until the score for each compound converged and did not improve further.

### Enzymes, Substrates, and Small Molecule Compounds

Recombinant ACE and ACE2 were obtained in purified form from R&D Systems, Minneapolis, Minn. (catalog ID: 929-ZN-10 and 933-ZN-10, respectively). Substrates for ACE (fluorogenic peptide V, Mca-RPPGFSAFK(Dnp)-OH, catalog ID: ES005), and for ACE2 (fluorogenic peptide VI, Mca-YVADAPK(Dnp)-OH, catalog ID: ES007) were also obtained from R&D systems. Top scoring molecules were obtained from the National Cancer Institute (NCI) for functional testing. Dry compounds were resuspended in 100% DMSO to prepare 100 mM stock solutions, according to the amount of compound provided by the NCI and its molecular weight. Gentle heating to 60-80 C was carried out to assist their solubilization. Some compounds were further diluted to 50 mM stocks if clearly difficult to dissolve.

### Activity Assays

Activity of ACE and ACE2 was measured with a Spectra Max Gemini EM Florescence Reader (Molecular Devices). The enzyme removes the c-terminal dinitrophenyl moiety that quenches the inherent fluorescence of its 7-methoxycoumain group, resulting in an increase in fluorescence in the presence of enzyme activity. Fluorescence was measured with excitation and emission spectra of 328 nm and 392 nm, respectively. Reaction mixtures were prepared in 100 μl volumes and different concentrations of compound were tested against 10 μM substrate. 10 nM enzyme in 100 mM NaCl, 75 mM Iris, 0.5 μM ZnCl₂, at pH 7.4. Samples were read every 15-20 seconds for at least 30 minutes immediately after the addition of fluorogenic peptide substrate at 37° C. Assays, including controls, were performed in the presence of 1% dimethyl sulfoxide (DMSO). Although higher concentrations of NaCl increase the activity of ACE2 and ACE (Vickers et al. 2002), a low concentration of salt (100 mM NaCl) was used in the assays to allow for enhancement of enzymatic activity to be detectable. That is, using 1 M NaCl which gives a maximal enhancing effect from the Cl ions might not allow the compounds to further enhance the activity of the enzyme. The lower salt concentration should give the compounds available room for activation.

Controls in the presence and absence of DMSO and without compound were carried out to evaluate the effect of DMSO on the activity of ACE and ACE2. Assays with no DMSO, 1% DMSO, and 2% DMSO were performed in identical conditions (i.e, pH, temperature, salt concentration, reaction mix volume and so on) to those of the experimental assays. At least up to 2% DMSO did not significantly affect the activity of ACE or ACE2 with the substrates used in this assay.

Active compounds were observed to absorb and emit background levels of fluorescence. The experimental assays were corrected at each concentration since higher or lower concentrations of compounds affected the background signal in a concentration dependent manner. The added or subtracted background levels from the active compounds, however, were constant throughout the duration of the assays and did not show increasing or decreasing background signals.

### Example 1

Approximately 140,000 compounds were virtually screened with DOCKv5.2 (Ewing et al. 2001) in 100 different orientations and ranked by energy score. This computer database was prepared with DOCK accessory software (SF2MOL2, UCSF) and Sybyl (Tripos, Inc.). Each compound was docked as a rigid body in up to 100 different orientations. The orientations were filtered by default bump filter parameters to exclude compounds with pronounced steric clashes. The grid-based scoring system was used for scoring with the non-bonded force field energy function implemented in DOCK. A standard 6-12 Lennard-Jones potential was used to evaluate van der Waals contacts. Spheres used by DOCK during matching algorithms were generated by SPHGEN.

Sites for molecular docking were identified by structural analysis in which the differences between the molecular surfaces of ACE2 in the open and closed conformation were calculated with DSSP (Kabsch and Sander, Biopolymers 22:2577-2637 (1983)). Three different molecular surface pockets, remote to the active site of ACE2, were selected with SPHGEN to dock and rank the compounds of the NCI database. Two sites were selected in the inhibitor bound form of the enzyme (sites 2 and 3, PDBID 14RL), and a single site was selected in the open conformation of ACE2 (site 1, PDBID 1R42). Structural analysis indicates that these surface sites are unique to only one of the two conformations.

After ranking with DOCK, the top scoring compounds for each site were tested in vitro with human recombinant ACE2 (R&D Systems). Active compounds were modeled bound to ACE2 with DOCK and were docked in at least 3,000 orientations, energy minimized, and with flexible bond parameters enabled. Other parameters such as number of minimization steps and number of conformations were also exhausted until the score for each compound converged and did not improve further.

The top ten scoring compounds for each of three sites are listed in Table 1. These compounds were requested from the National Cancer Institute, Developmental Therapeutics Program (NCUDTP) for functional testing and are identified by their NSC catalog number. The top ten scoring compounds of each site share some general characteristics. Site 1 clearly selected for uncharged smaller compounds with relatively few hydrogen bond donors and acceptors. The average molecular weight of the top ten scoring compounds is 279 Da. The x Log P values seem to range from 0.75 to 3.38 for most compounds of site 1 and a single compound (no. 8) seems to slightly violate the Lipinski “rule of 5” (MW<500, c Log P <5, H-bond donors <5, H-bond acceptors <10) in this regard (Lipinski et al. 1997). The Lipinski rule of 5 states that compounds are likely to have poor absorption and permeation when two or more parameters are out of range. In contrast to the compounds selected for site 1 by DOCK, sites 2 and 3 seem to meet the Lipinski criteria less conservatively. Site 2 favored neutral or negatively charged compounds of a slightly larger molecular weight (MWₐᵥₑ351 Da) and c Log P values have a wider range from −4.35 to 5.33.

For both site 2 and 3 most compounds have a higher number of hydrogen bond donors and acceptors, with many exceeding cut off criteria. Both of these sites also selected for compounds with a higher number of rotable bonds. Follow up studies to those of Lipinski favor molecules that have less than 7 rotable bonds as this may be another factor that affects the druglikeness of small molecules. Site 3 seems to have favored positively charged compounds of an even higher molecular weight (MWₐᵥₑ 435 Da) compared to site 1. Most compounds in the top ten list for site 3 do not meet Lipinski criteria in at least one parameter. The shared characteristics of these compounds likely reflect the properties of the sites selected for virtual screening and it appears site 1 is better fit for the ligation of a druglike molecule.

TABLE 1
Table 1 shows the top ten scoring compounds for the three different sites docked. All
requested from the NCI/DTP for in vitro testing.

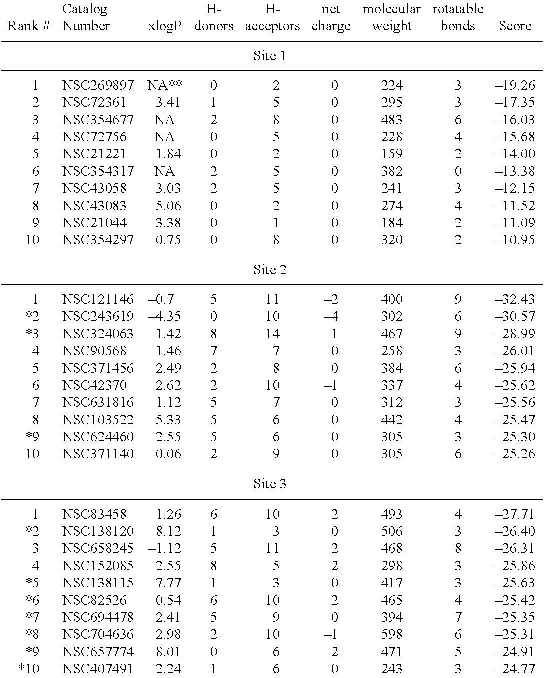

*Not obtained from the NCI.
**Not Available.
Active compounds are highlighted.

Only 21 of the requested compounds were obtained and tested for enhancement of ACE2 activity. Dry compounds were dissolved initially in 100% DMSO, and dilutions were obtained from this solution. During initial functional screening, compounds 3 and 6, both selected for site 1, were observed to increase ACE2 activity about 2-fold. Both compounds share some structural similarities, each including a rigid ring system scaffold with hydrogen bond donors. Both compounds show a multicyclic scaffold that was docked in approximately the same orientation. In the best scoring orientations for each compound, hydrogen bonding donors and acceptors occur in both compounds at similar positions

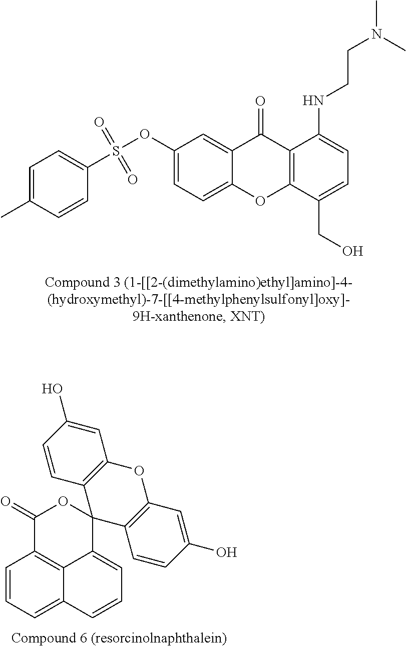

These observations demonstrate consistency in the in silico simulations. They show that DOCK was able to select two different but similar compounds that presumably interact with the same site and have similar activities out of an in silico library of ˜140,000 compounds.

### Example 2

Human recombinant ACE and ACE2 were obtained from R&D systems, Minneapolis, Minn., along with their respective fluorogenic substrates (ACE, catalog ID: 929-ZN-10; ACE2, 933-ZN-10; ACE substrate, fluorogenic peptide V, Mca RPPGFSAFK(Dnp)-OH, catalog ID: ES005; ACE2 substrate, fluorogenic peptide VI, Mca-YVADAPK(Dnp)-OH, ES007). Enzymatic activity was measured with a Spectra Max Gemini EM Fluorescence Reader (Molecular Devices) (Huentelman et al., Regul. Pept. 122:61-67 (2004)). Compounds were tested against 50 μM substrate. All assays were performed at least in triplicate in a reaction mixture containing 10 nM enzyme, 1 M NaCl, 75 mM Tris-HCl, 1% DMSO and 0.5 μM ZnCl2, at pH 7.4. Samples were read every 15-30 seconds for at least 30 minutes immediately after the addition of fluorogenic peptide substrates at 37° C. DMSO did not affect the activity of ACE or ACE2 under these conditions. Enzyme activity was corrected for background.

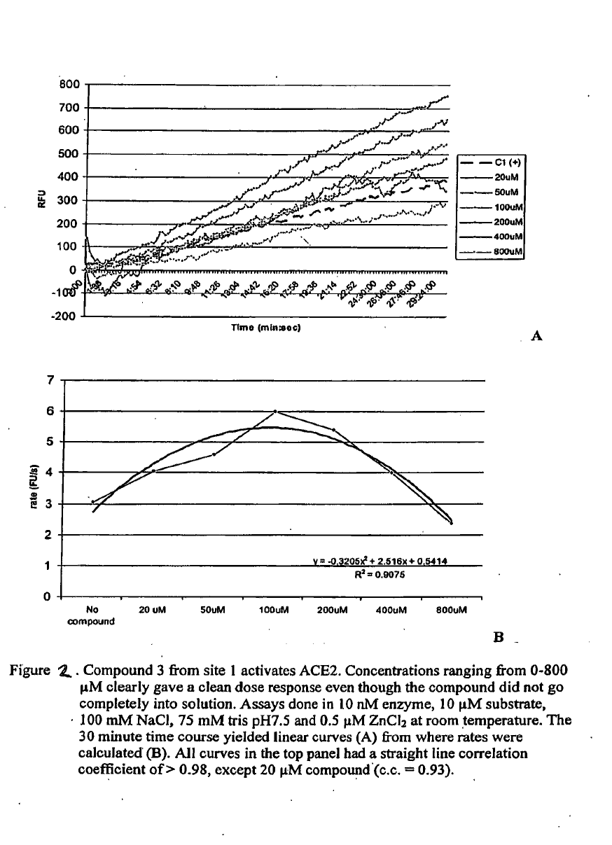

Compounds 3 and 6 were assayed again to confirm their effect on ACE2 activity. They were confirmed to enhance enzymatic activity 2-fold and both compounds have similar activity profiles across a wide concentration range. All assays were performed in 1% DMSO. Control experiments showed that 1 and 2% DMSO did not affect ACE2 activity in the absence of compounds. Compound 3 showed a maximum activation at 100 μM with a clean dose response that almost doubled ACE2 activity at 100 μM compound (FIG. 2). At concentrations higher than 100 μM however, Compound 3 became inhibitory with 400 μM returning enzymatic activity to approximately control levels and with 800 μM inhibiting its activity slightly below that of control.

This inhibition at such high concentrations may be a consequence of compound aggregation, which is known to promiscuously inhibit enzymes by sequestering the enzyme from solution. Another artifact that could possibly occur under the conditions of our assays is related to the coordination of zinc by the large number of lone pairs of electrons from the active compounds. Oxidized zinc may be coordinated by these compounds at high concentrations. Although metalloproteases usually have a high affinity for their metals, 0.5 μM zinc may be a low concentration of zinc compared to 500 and 800 μM compound. Finally, these high concentrations of compound may force them to bind the enzyme at secondary low affinity sites that may still modulate the activity of the enzyme (e.g., to inhibit it). However it should be noted that the rates of enzyme activity obtained from these spectrophotometric assays (RFU/s) across this wide concentration range (0-800 μM) approximate a quadratic curve closely (FIG. 2) and that this inhibition may still be consistent with a conformational equilibrium shift mechanism. In the case of the latter a high concentration of activator may still prevent the enzyme from shifting into the closed form of this enzyme, if indeed the compound is found to stabilize the open form. Although the overall inhibition observed for compound 3 in FIG. 2 may not be significant when compared to control activity, the rates of enzyme activity give a clear dose response pattern on the ACE2 modulating effects of compound 3.

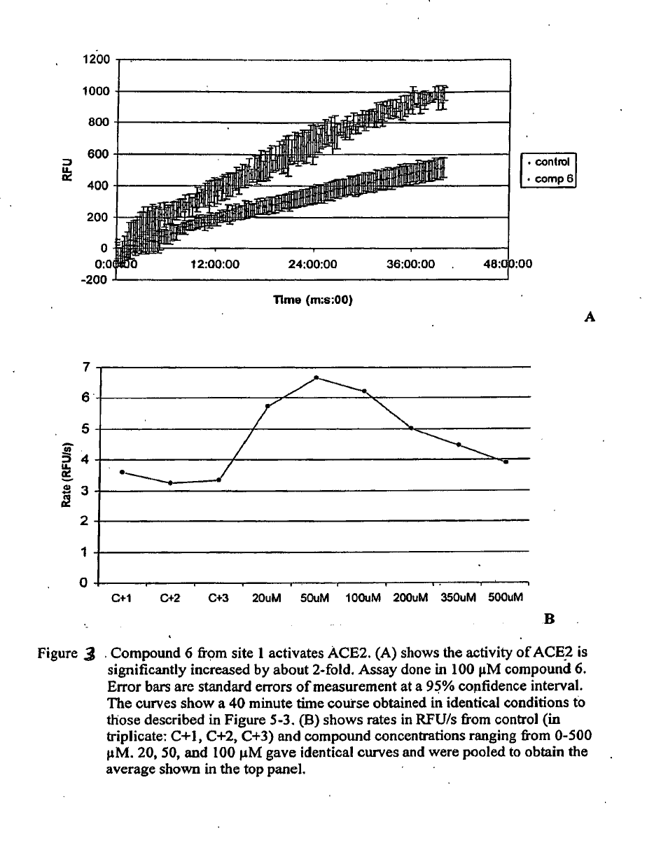

Compound 6 did not show the same dose response but activated ACE2 similarly (FIG. 3). Compound 6 activated ACE2 identically at 20, 50 and 100W but like compound 3 it inhibited ACE2 at higher concentrations. At 500 μM compound 6 ACE2 activity returned down to control level. It is observed that compound 6 was significantly more insoluble than compound 3 and the lesser quality of the data may be a reflection of its poor solubility. One explanation to the equal activating effect of compound 6 on ACE2 at different concentrations (20, 50 and 100 μM) would be that compound 6 has already reached its maximum effect at 20 μM, and that raising the concentration of the compound further only forms more aggregate. The effective concentration of compound 6 available in solution would be the same at all concentrations. In this case it is likely that the inhibition observed is due to aggregate and may be nonspecific. Lower concentration titrations would be necessary to reveal a clearer dose response but the effect may be too weak to observe with confidence.

Overall, Compound 3 seems to behave more promisingly. Both compounds appear to be relatively non-toxic. The National Cancer Institute provides that both were tested in anticancer screens and more than 95% of rats subjected to 200 mg/Kg of compound had survived after 30 days of exposure. Since XNT is significantly more soluble than resorcinolnaphthalein, it was selected for large scale synthesis and in vivo testing.

### Example 3

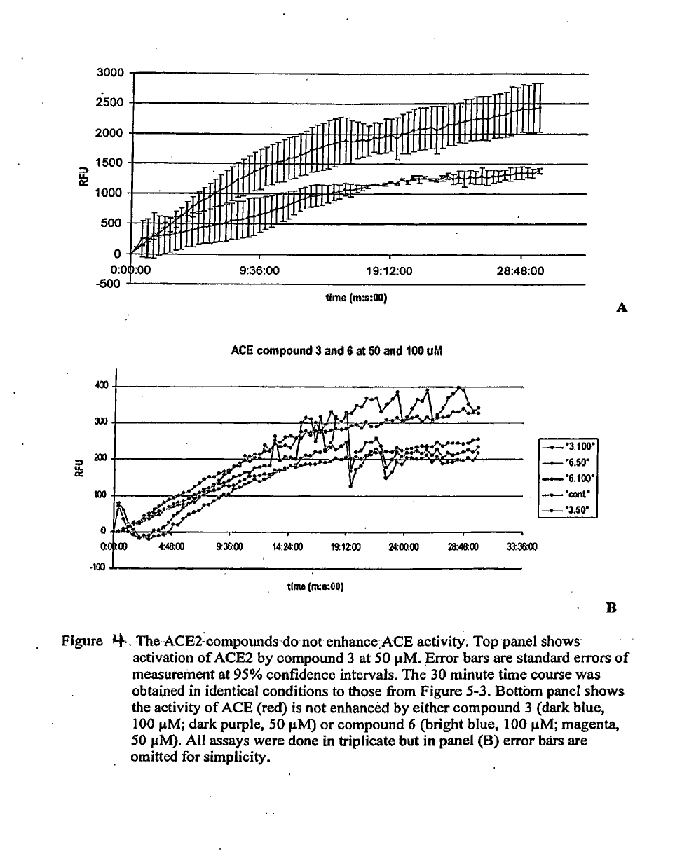

Compounds were tested in similar conditions for ACE activation. As shown in FIG. 4, compounds 3 and 6 did not activate ACE at either 50 or 100 μM. ACE is 42% homologous to ACE2 and is also activated by chloride ions. These experiments support that compounds 3 and 6 selected by virtual screening methods targeting the open form of ACE2 have a specific measurable enhancing effect on enzymatic activity.

These results suggest that a structure-based approach to the identification of remote site activators could also be applied to the discovery of new inhibitors for other enzymes.

### Example 4

XNT was dissolved in saline at low pH (2-2.5) for in vivo studies. This compound was consistently prepared 24-48 hours before delivery in animals. XNT was prepared on a gram scale in six synthetic steps from 5-methoxysalicylic acid and m-chloroiodobenzene through modifications of a published procedure (Archer et al., J. Med. Chem. 26:1240-1246 (1983); Archer et al., J. Med. Chem. 31:254-260 (1988).

Animal Procedures

All animal procedures were performed in compliance with approved IUCAC protocols and regulations. WKY rats were purchased from Harlan Sprague Dawley, Inc (Indianapolis, Ind., USA). SHR rats were purchased from Charles River Laboratories (Wilmington, Mass., USA). All rats were 8 week old (200-225 g) males.

Indirect blood pressure was measured weekly as previously described (Iyer 1996, Lu 97). Rats were acclimated to the procedure before data collection with a programmed Electro-Sphygmomanometer (Narco Bio Systems, Austin, Tex., USA) and a PowerLab signal transduction unit (ADInstruments, Colorado Springs, Colo., USA). Data was recorded and analyzed electronically with Chart. The systolic blood pressure for each animal is the average of at least 5 separate measurements.

For direct blood pressure measurements, a polyethylene cannula (PE-50, Clay Adams) was implanted in the carotid artery as preciously reported (Lu 1997). Similarly, a silicone elastomer cannula (PE-10, Helix Medical) was implanted in the jugular vein for acute intravenous drug administration. Animals were anesthetized with a mixture of ketamine, xylazine, and acepromazine (30, 6, and 1 mg/kg, respectively) and were allowed 24 h to recover. Blood pressure responses to acute injections of Compound 3 (10 mg/kg) in awake, freely moving animals were recorded.

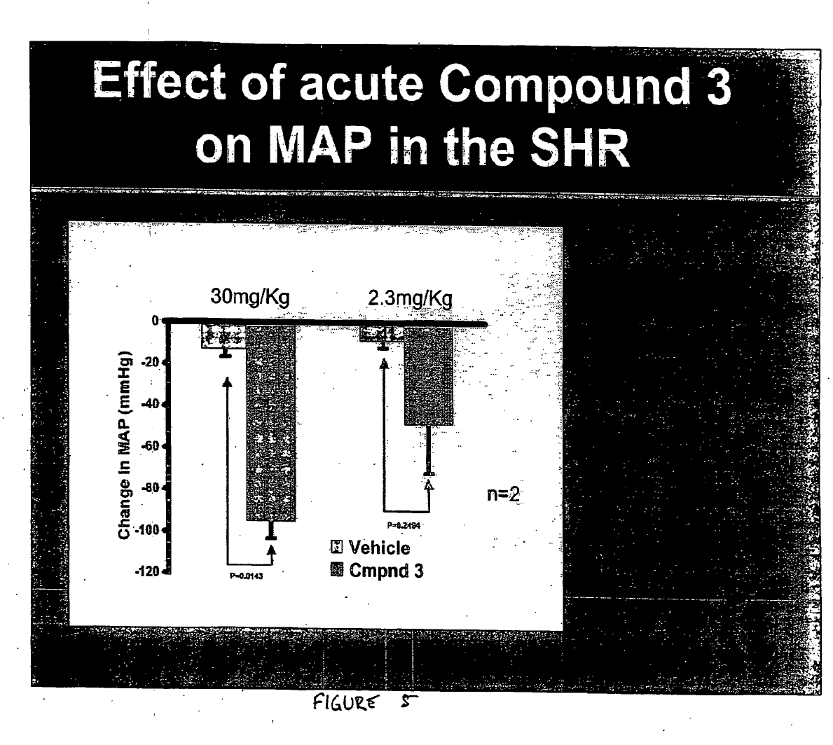

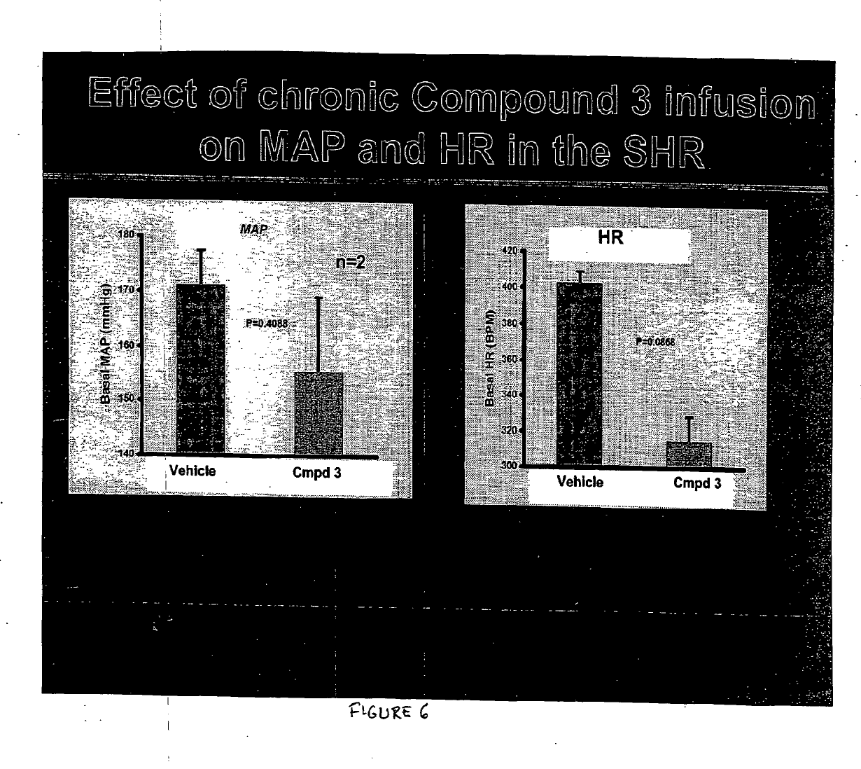

As seen in FIGS. 5 and 6, infusion of the compound results in a decrease in mean arterial pressure (MAP) in SHR rats when administered acutely (FIG. 5) or chronically (FIG. 6). A decrease in heart rate (HR) was also seen FIG. 6).

### Additional Methods

Male WKY rats and SHR of 14-16 weeks of age (300-325 g body weight) were purchased from Charles River Laboratories (Wilmington, Mass., USA).

Acute hemodynamic measurements. Mean arterial pressure (MAP) and heart rate (HR) were continuously monitored in SHR and WKY animals (n=3-9) fitted with both a jugular and carotid cannulae. Briefly, animals were anesthetized with a mixture of ketamine, xylazine, and acepromazine (30, 6, and 1 mg/kg, respectively). A polyethylene cannula (PE-50, Clay Adams) was introduced into the carotid artery for direct BP measurements, while a silicone elastomer cannula (Helix Medical) was introduced into the descending jugular vein for acute intravenous injections of drug. Both cannulae were filled with heparin saline (40 U/mL, sigma), and sealed with stylets. Dose-response curves were obtained in awake, freely moving animals after a 24-48 hour recovery period. Doses of XNT (0.5, 1, 5, and 10 mg/Kg) were applied as a bolus administration via the jugular cannula and BP and HR data was recorded and interfaced to a PowerLab (ADInstruments) signal transduction unit. Data were analyzed using the Chart program supplied with the PowerLab system.

Chronic hemodynamic measurements. Osmotic minipumps (Alzet, model 2004) containing either 10 mg/ml XNT (60 μg/day, 28 days, n79) or vehicle (saline, pH 2-2.5) were implanted subcutaneously after allowing them to equilibrate in sterile saline at 37° C. for 24 h. XNT was delivered at an infusion rate of 260 ng/Kg/min. BP was measured indirectly by the “tail-cuff” method in conscious animals every week for 4 weeks.

After 28 days of saline or XNT infusion, animals were cannulated as described above and acute hemodynamic responses to Ang II (5, 10, 20, 40, 80, and 160 ng/Kg), bradykinin (BK) (0.06, 0.6, 6, 14, and 28 ng/Kg) and losartan (0.25 mg/Kg) were measured in both WKY rats and SHR.

Isolated heart preparation. After analysis of the BP responses to Ang II, BK, and losartan, animals were allowed to recover for 24 hours. An intraperitoneal injection of heparin (400 IU) was administered to each animal. Ten to fifteen minutes later, the hearts were dissected and perfused according to the Langendorff technique. Briefly, hearts were perfused through an aortic stump with Krebs Ringer solution containing 118.4 mM NaCl, 4.7 mM KCl, 1.2 mM KH2PO4, 1.2 mM MgSO4.7H2O, 2.5 mM CaCl2.2H2O, 11.7 mM glucose, and 26.5 mM NaHCO3. The perfusion flow was maintained constant (8-10 ml/min) at 37° C. along with constant oxygenation (5% CO2 and 95% O2). Intraventricular pressure and coronary perfusion pressure were continuously recorded using a PowerLab signal transduction unit (ADInstruments, Colorado Springs, Colo., USA). After 20 to 30 minutes of stabilization, functional parameters were recorded for an additional period of 30 minutes. Data from vehicle or XNT-treated animals was analyzed electronically with Chart software.

Histological analysis. At the end of the chronic study, hearts and kidneys were fixed in 10% buffered formalin, embedded in paraffin, and sectioned to a thickness of 5 μm. Sirius red staining was carried out to assess the extent of collagen deposition. Cardiac and renal interstitial fibrosis at 100× magnification was measured by percent area analysis. Perivascular fibrosis was measured at 250× magnification and data was normalized to vessel lumen. An Olympus BX 41 microscope was used for imaging and quantification of collagen density data was carried out with ImageJ software from the NIH.

Immunohistochemistry and immunocytochemistry staining. Heart sections from SHR and fibroblasts in culture from adult rat hearts were used to assess the effects of XNT on Ang-(1-7) and ACE2 immunoreactivities. Five micron sections from hearts were fixed as described above and fibroblasts were fixed with 4% paraformaldehyde for 15 min at room temperature. Nonspecific binding sites were blocked with normal goat serum diluted in PBS (1:70) and endogenous peroxidase with 3% H2O2 in PBS for 1 h. Sections were incubated overnight at 4° C. with one of the following primary antibodies: rabbit anti-rat polyclonal Ang-(1-7) (1:600) or rabbit anti-rat polyclonal ACE2 (1:500; GeneTex, Inc.). Antibody specificity was previously established 21, 22. After 2-3 rinses in PBS, the sections were incubated with biotinylated anti-rabbit antibody for 1 h at room temperature (1:200; Vector Laboratories). Following PBS rinses, sections were incubated with ABC reagent (avidin-biotinylated enzyme complex; Vector Laboratories) for an additional 1 h at room temperature and stained brown with a solution containing 3,3′-diaminobenzidine tetrahydrochloride (Vector Laboratories). Sections were mounted using VectaMount (Vector Laboratories). Negative controls were obtained by omission of primary antibodies. Fibroblasts in primary culture were processed essentially as described for heart sections except 0.6% H2O2 was used to block endogenous peroxidase. To treat fibroblasts, 100 μM XNT was added directly to culture media and incubated for 1 hour. Immunoreactivity quantification was performed according to published methods.

Statistical analysis. Data are expressed as mean±SEM. Unpaired Student's t-test and 1-way ANOVA were performed for statistical analysis. For cardiac function, response to Ang II, BK, and losartan experiments, statistical significance was estimated using 2-way ANOVA followed by the Bonferroni test. Differences were considered significant at a p<0.05 or p<0.001, as indicated. Tests were performed with the PRISM software package from GraphPad, San Diego.

### Example 5

### Effects of XNT on Blood Pressure

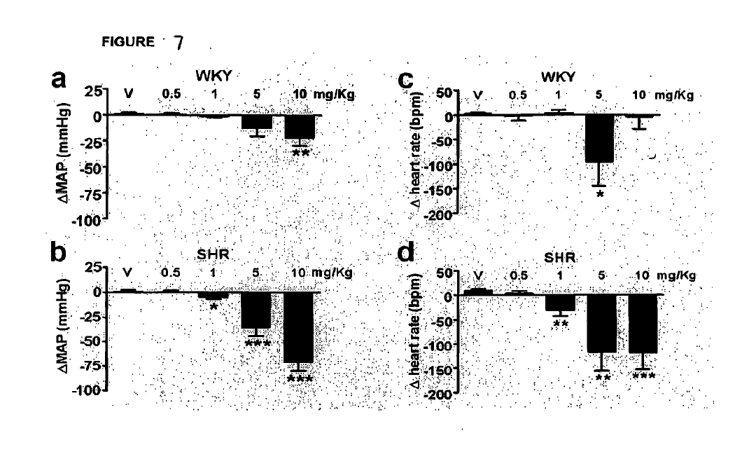

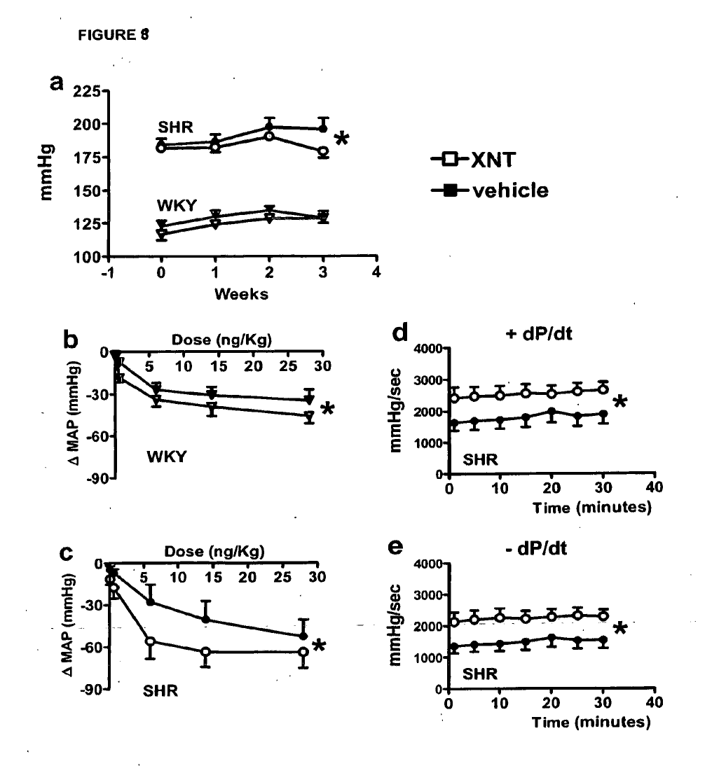

Acute intravenous injections of XNT resulted in a rapid and transient decrease in BP (FIG. 7a, 7b). It caused a significant decrease in BP in the SHR with a dose as low as 1 mg/Kg. A maximal decrease of 71±9 mmHg on BP was observed with 10 mg/Kg (FIG. 7b). Decreases in BP were accompanied by decreases in HR (FIG. 7c, 7d). In contrast to SHR, XNT had no significant effect on WKY rat BP with 1 mg/Kg and showed only modest decreases in BP with 5 and 10 mg/Kg. Thus, the antihypertensive effect of XNT was significantly more pronounced in the SHR compared to WKY rats (FIG. 7a, 7b). Compared to the 71±9 mmHg decrease observed in the SHR, only a 21±8 mmHg decrease was observed in WKY rats with a dose of 10 mg/Kg (p<0.05). Sprague-Dawley rats showed a response to XNT that was similar to WKY rats (data not shown). In addition, vehicle alone did not show any significant effects on BP or HR in either strain of rats. Chronic infusion of XNT produced a significant reduction in the BP of SHR, but not in WKY rats. The decrease in BP during xanthenone infusion was gradual and it achieved the maximal effect (17 mmHg, 2-way ANOVA, p<0.05) by the third week (FIG. 8a).

Since ACE2 is involved in the metabolism of angiotensin peptides and kallikreinkinin system (KKS) peptides, the BP responses to acute administration of BK, Ang II and to the Ang II type-I receptor antagonist, losartan, were evaluated in WKY rats and SHR after 4 weeks of XNT infusion. BK-induced decreases in BP were more pronounced in XNT-treated WKY rats and SHR (FIG. 8b, 8c). Also, the potentiation of this BK hypotensive effect in XNT-treated rats was significantly greater in the SHR (FIG. 8c) compared to WKY rats (FIG. 8b) (43±12 mmHg vs. 28±8 mmHg, p<0.05). However, no significant differences in Ang-II-induced increase or losartan-induced decrease in BP were observed between saline and XNT-treated WKY rats and SHR (data not shown).

In addition, XNT effects on cardiac function were analyzed using the Langendorff preparation. Chronic infusion of XNT resulted in an increase in +dP/dt and −dP/dt in SHR (FIG. 8d, 8e). No significant changes were observed in left ventricular systolic pressure, left ventricular end diastolic pressure, perfusion pressure, and HR. XNT had the same significant effect on the cardiac function of WKY rats (data not shown).

### Example 6

### Effects of XNT on Cardiac and Renal Fibrosis

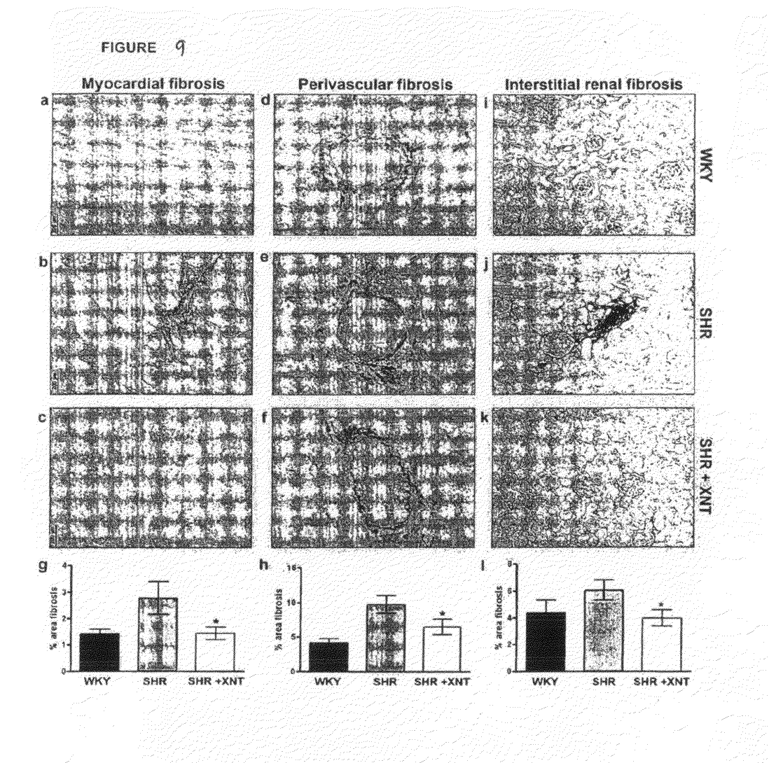

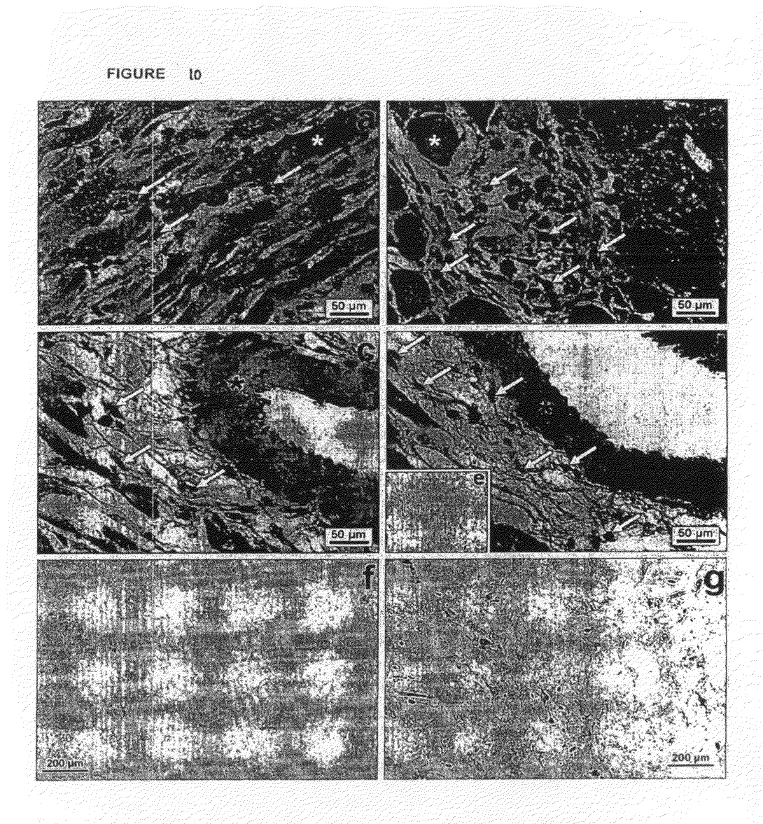

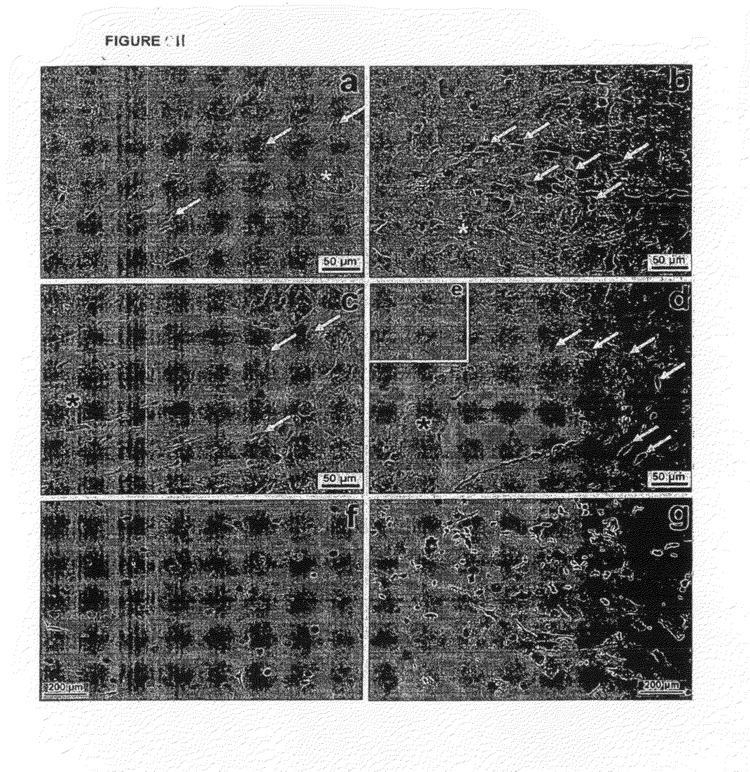

The effect of chronic XNT infusion on cardiac and renal fibrosis was examined. Chronic XNT treatment caused a significant reversal of both myocardial and perivascular fibrosis in the SHR heart (FIG. 9a-9h). Similarly, a significant reversal in renal interstitial fibrosis was observed in SHR chronically treated with XNT (FIG. 9i-91). Since Ang-(1-7) is the major product of ACE2 29 and since Ang-(1-7) has been shown to be antifibrotic, we determined if XNT treatment resulted in increases in Ang-(1-7) and ACE2 levels in hearts from SHR. Endogenous Ang-(1-7) and ACE2 immunoreactivities were present in cardiomyocytes (FIG. 10a, 10c and FIG. 11a, 11c). In addition, Ang-(1-7) and ACE2 immunoreactivities were also observed in cardiac fibroblasts (FIG. 10a, 10c and FIG. 11a, 11c). Chronic infusion of XNT, which causes a decrease in the collagen content in SHR (FIG. 9), resulted in ˜16% increase in the number of cardiac fibroblasts, but not cardiomyocytes, that stained positive for Ang-(1-7) and ACE2 (FIG. 10b, 10d and FIG. 11b, 11d). Additionally, the intensity of the Ang-(1-7) and ACE2 staining was also increased in cultured adult fibroblasts treated with XNT (100 μM) (FIG. 10g and FIG. 11g). In contrast, plasma levels of Ang-(1-7) did not change (28.4±4.4 vs. 23.7±3.3 pg/ml in XNT-treated SHR, n=7-9). These findings indicate that the antifibrotic effects observed in the XNT-treated rats may be mediated by an increased local ACE2 activation and production of Ang-(1-7).

### Discussion

Both compounds 3 and 6 are predicted to interact with the same site of ACE. This suggests that we have discovered a molecular surface pocket outside of the active site of an enzyme capable of modulating enzymatic activity upon ligation by a small molecule. This is a striking result considering we have limited ourselves to functionally test only the top ten scoring compounds for each site (typical drug discovery campaigns test thousands of compounds) and that we only screened three sites on ACE2. Clearly virtual screening methods as implemented by DOCK serve to increase the efficiency of initial screening assays. The results reported here show that these compounds are selective for ACE2 and do not enhance ACE activity, which is 42% identical to ACE2 (i.e., their catalytic domains).

Site 1 clearly selected for a group of compounds that meet druglikeness criteria (Lipinski et al. 1997). Compared to sites 2 and 3, the characteristics of these compounds may reflect properties of the molecular surface site on which they were screened. Out of a library of ˜140,000 compounds, the top ten compounds for each site shared a group of physicochemical characteristics (Table 1). In aiming to identify remote sites from the active site an enzyme that could potentially be exploited for drug development, it may be desirable for these sites to not only have unique features among different conformers, but also have characteristics that are likely to favor ligation of a druglike molecule. Similar to Lipinski rules of 5 now commonly used to pre-screen small molecules, there may be a set of criteria we could follow when selecting a molecular surface pocket to probe. For example, the size of the pocket will limit the size of small molecules since DOCK will eliminate compounds that do no fit into a pocket. Smaller molecules would in turn be less likely to have too many hydrogen bond donors or acceptors. However, selecting a site that is too small may leave no room for lead optimization.

Compound 3 and 6 were docked with minimization while treated as flexible ligands to obtain the most accurate prediction of their complex with ACE2. The three-member ring scaffold is positioned similarly in site 1 for each compound. Both compounds are predicted to engage in several hydrogen bonds with residues from ACE2, although the hydrogen bonding interactions do not involve the same residues.

According to DOCK, compound 3 hydrogen bonds with residues Lys94, Tyr196, Gly205 and His195. The NZ nitrogen from the lysine side chain is positioned at 3.25 A from the hydroxyl group oxygen (O3) in compound 3. The carbonyl oxygen (O2) is within 3.16 A from the hydroxyl group of Tyr196. The distal amine nitrogen (N2) from compound 3 interacts at a distance of 3.31 Å with the main chain carbonyl oxygen of glycine in ACE2. And the ND1 nitrogen from the ACE2 histidine is within 2.98 and 3.31 A of the ether-sulfate oxygens (O4 and O6 respectively) in compound 3. All hydrogen bonding angles show good geometry (125-130°), except for the angle C14-O3-NZ which is wider(160°). Given that lysine side chains are very flexible, however, an experimental structure is likely to show the side chain of Lys94 oriented in a more favorable orientation.

Compound 6 seems to be involved in 3 hydrogen bonds with residues Gln98, Gln101 and Gly205. Both hydroxyl oxygens in compound 6 interact with main-chain carbonyl oxygens of ACE2; 05 seems to bond to Gly205 (3.18 A) and O4 to Gln101 (3.33 Å). The ester oxygen (O2) in compound 6 accepts an amide hydrogen from the side chain of Gln98 at a slightly less ideal distance of 3.51 A, but as mentioned for the model of compound 3, docking simulations do not account for any “induced fit” effects on ACE2 residues. An experimental structure is likely to show better hydrogen bonding distances and geometry for both compounds. At present it is nonetheless observed that 3.5 A is an acceptable hydrogen bonding distance. Like for compound 3, hydrogen bonding angles are as expected (˜117°).

If the compounds identified in this study interact with the open conformation of ACE2 at site 1, they may specifically stabilize this conformation in solution. Without wishing to be bound by any theory, this effect may enhance ACE2 activity by at least two mechanisms. Logically, closed conformations of the free enzyme do not allow substrate into its active site. In the presence of compound, the populations of free enzyme may be shifted to that of the open form effectively increasing the activity Coefficient of the enzyme. Alternatively, it is also possible that product release is a rate limiting step in ACE2 turnover. This is known for several enzymes (e.g., dihydrofolate reductase, also mentioned above). The activity of ACE2 in the presence of compound may then be enhanced as the enzyme-product complex empties more quickly and ACE2 becomes available to start another cycle. It is possible that compounds 3 and 6 modulate ACE2 activity by both mechanisms. In both cases, compounds would be acting by shifting the populations of enzyme into a conformation that is fully active, whether the enzyme is in free or bound form, and helping the enzyme avoid “wasting its, time” on nonproductive complexes or conformations.

In this study, we begin to test the hypothesis that targeting a specific enzyme conformation with small drug-like molecules will enhance enzymatic activity by shifting the conformational equilibrium of the enzyme favorably for its activity. This hypothesis is based on recent enzyme structure, dynamic and kinetic data demonstrating that conformational changes involved in binding or release of ligands may be rate limiting. Importantly, the monovalent anion-dependent enhancement of activity observed for our model enzyme, ACE2, has been suggested to occur by this mechanism. For hinge bending enzymes, such as ACE2, the large conformational change that opens and closes their active site allows for a unique opportunity to measure the effects of targeting specific enzyme conformations in a key protein involved in regulating BP and CVD (FIG. 1). In this study, we attempt to understand how drug-like molecules can be developed to probe protein dynamics and enhance enzyme activity. A similar approach may be applied to develop novel enzyme inhibitors targeted away from the active site (i.e. conformational equilibrium could be shifted in the opposite direction). This strategy would be useful for targeting enzymes resistant to current therapeutics such as HIV protease or enzymes expressed in multi-drug resistant pathogens (e.g. amidase in tuberculosis).

With these considerations in mind, more than 140,000 small molecules were molecularly docked into structural pockets present in crystal structures of ACE2 in the open and closed conformations (Table 1). Selected compounds were tested in vitro and allowed us to identify two active compounds directed at a structural pocket present in the open conformation of ACE2: XNT and resorcinolnaphthalein. Both compounds enhanced ACE2 activity in a dose-dependent manner and were ACE2-specific, as they did not significantly affect ACE activity (FIG. 1). These data demonstrate the selective strength of this novel approach in pinpointing specific structural pockets and conformations since the ACE2 and ACE catalytic domain share 42% sequence identity.

The observation that 20% of the compounds directed at site 1 function in enhancing ACE2 activity whereas no compounds directed at sites 2 and 3 enhance enzyme activity suggests that the structural pocket defined by site 1 in the open conformation may be a valid target for therapeutic development (FIG. 1c, 1e). Structural analysis shows that both conformations of ACE2 have 10-15 surface pockets with adequate solvent accessible volumes (DSSP and castP) but only a few of these sites are unique to one specific conformation. This structure-based approach is distinctly different from those employed in previous efforts because multiple specific enzyme conformations were targeted distal to the active site with the goal of enhancing enzyme activity.

A significant observation in this study is that XNT, a compound that enhances ACE2 activity, causes considerable reductions in BP and a striking reversal of cardiac and renal fibrosis in the SHR model of HT. This observation is remarkable because rational drug design is traditionally directed at the discovery of enzyme inhibitors or receptor blockers that compete with the natural ligand. Here, we present for the first time a structure-based drug-discovery approach to enable rational development of enzyme activators. In addition, we identified a compound that, for the first time, results in a beneficial outcome on both BP and tissue remodeling in the heart and kidney. The clinical ramifications of this study are directly significant for CVD and diseases associated with hypertension, such as obesity and diabetes. Moreover, we define a novel rational drug design strategy to address new challenges in the prevention and treatment of human diseases.

We selected XNT for in vivo studies because of its more favorable solubility properties for administration. Bolus injection of XNT caused a dose-dependent decrease in BP (FIG. 7), which was significantly more pronounced in the SHR compared with WKY and SD rats. XNT also induced a significant decrease in HR. This effect could be the consequence of a direct action of XNT in the heart, direct change in the autonomic activity (increase vagal/decrease sympathetic tonus) or changes in the set-point of the baroreflex at the central nervous system (CNS). An effect on the CNS is consistent with observations after overexpression of ACE2 in the RVLM, which resulted in a marked decrease of BP and HR in SHR. It is important to note that XNT did not elicit any changes in the HR of isolated hearts. However, we cannot exclude a direct effect of XNT on HR because isolated heart perfusion was performed after 4 weeks of systemic XNT infusion and not directly with a solution containing XNT. More importantly, chronic infusion of XNT also induced a reduction in the BP of SHR, but did not alter HR. The unaffected HR in this protocol was probably due to the different approaches utilized (acute vs. chronic administration) and the final effective plasma concentration of XNT after acute and chronic treatment.

Consistent with the beneficial effects of ACE2 activation on BP, we found that cardiac function is improved in isolated hearts after chronic infusion of XNT in the SHR (FIG. 8). The mechanism of this improvement remains to be elucidated; however, an indirect effect as a result of the decrease in BP is a possibility. Since XNT-treated SHR also presented a reversal in myocardial and perivascular fibrosis (FIG. 9), the improvement in heart function is more likely due to the marked reduction in collagen deposition in cardiac tissue. In fact, if after ACE2 activation there is an increased Ang-(1-7) production with concomitant degradation of Ang II, this hypothesis is plausible, since Ang II is a pro-fibrotic peptide 22 and Ang-(1-7) possesses anti-fibrotic actions. This conclusion is consistent with our immunohistochemical data indicating that Ang-(1-7) and ACE2 immunoreactivity was increased in cardiac fibroblasts of SHR treated with XNT (FIGS. 10 and 11). In addition, incubation of primary cultured cardiac fibroblasts with XNT in vitro causes significant increases in Ang-(1-7) and ACE2 immunostaining. The anti-fibrotic effect of XNT was not limited to the heart, because it also reversed interstitial fibrosis in kidneys of SHR (FIG. 9).

As anticipated, the hypotensive effect of BK is more pronounced in SHR than in WKY rats (FIG. 8). Furthermore, we observed that XNT infusion potentiates the BK response in WKY rats and SHR. Again, these data suggest that the XNT actions may be, at least partially, mediated by an increased Ang-(1-7) production, since it has been demonstrated that Ang-(1-7) potentiates the hypotensive effect of BK in previous preparations.

In conclusion, we successfully identified a compound that enhances ACE2 activity in vitro and shows anti-hypertensive and cardioprotective effects along with reversal of both cardiac and renal fibrosis.

In addition, we report not only the identification of ACE2 activators, but also a novel structure-based drug discovery approach that may be applicable to other enzymes, by targeting allosteric sites on the molecular surface of enzymes to enhance or inhibit their activity. Enzymatic activators are rare and their development by current structure-based knowledge is unprecedented. Identification of molecular surface sites remote from the active site of the enzyme can be exploited for drug development. This approach may open new doors in drug therapy as the identification and design of activators becomes a tractable route. This will expand the availability of macromolecular targets and also offer hope for the development of novel inhibitors for enzymes resistant to current therapeutics.

Increased ACE2 activity represents an alternative strategy for the treatment of hypertension, pulmonary hypertension, and related cardiovascular and cardiopulmonary diseases. The monovalent anion-dependent enhancement of ACE activity, similarly observed for ACE2 (Vickers et al. 2002), has been suggested to occur by this mechanism and is consistent with kinetic studies on the effect of chloride ions on ACE (Towler et al. 2004). Therefore, the crystal structures of the open and inhibitor bound forms of ACE2 were analyzed to identify molecular surface features unique to each conformation. Virtual screening methods were applied to identify small molecules capable of enhancing ACE2 activity. Molecular surface sites remote to the active site were targeted and 2 compounds able to increase enzymatic activity 2-fold were identified. Both compounds are predicted to bind at the same site and share structural similarities. Furthermore, these compounds clearly enhance ACE2 activity while not affecting ACE activity (see the Examples, infra). To date it appears this is the first report of in silico docking and structure-based approach used to identify enzymatic activators.

Additional ACE2 activators can be identified by the methods described herein, and improvements on those methods. For example, physical interactions of these compounds with ACE2 can be analyzed to validate molecular docking simulations. Crystallization conditions for ACE2 are known. Solving the structure of ACE2 bound to the active compounds will confirm their site of interaction, orientation and specific interactions involved.

### REFERENCES

Agrawal A., Eastman Q. M. and Schatz D. G. (1998) Transposition Mediated by RAG1 and RAG2 and its Implications for the Evolution of the Immune System. Nature 394, 744-751.
Asensio J. L., Canada F. J., Bruix M., Rodriguez-Romero A. and Jimenez-Barbero J. (1995) The Interaction of Hevein with N-Acetylglucosamine-Containing Oligosaccharides. Solution Structure of Hevein Complexed to Chitobiose. Eur. J. Biochem. 230, 621-633.
Ash E. L., Sudmeier J. L., Day R. M., Vincent M., Torchilin E. V., Haddad K. C., Bradshaw E. M., Sanford D. G. and Bachovchin W. W. (2000) Unusual 1H NMR chemical shifts support (His) C(epsilon) H-bond: proposal for reaction-driven ring flip mechanism in serine protease catalysis. PNAS 97, 10371-10376.
Atkinson M. A. and Leiter E. H. (1999) The NOD Mouse Model of Type 1 Diabetes: As Good as it Gets? Nat. Medicine 5, 601-604.
Babu M. M. (2003) NCI: A Server to Identify Non-Canonical Interactions in Protein Structures. Nucleic Acids Res. 31, 3345-3348.
Bader M. (2002) Role of the Local Renin-Angiotensin System in Cardiac Damage: A Minireview Focussing on Transgenic Animal Models. J. Mol. Cell. Cardiol. 34, 1455-1462.
Bajorath J. (2002) Integration of Virtual and High-Throughput Screening. Nat. Rev. Drug Discov. 1, 882-894.
Barclay A. N. (2003) Membrane Proteins with Immunoglobulin-Like Domains—A Master Superfamily of Interaction Molecules. Semin. Immunol. 15, 215-223.
Barton G. J. (1993) ALSCRIPT: A Tool to Format Multiple Sequence Alignments. Protein Eng. 6, 37-40.
Becker O. M., Marantz Y., Shacham S., Inbal B., Heifetz A., Kalid O., Bar-Haim S., Warshaviak D., Fichman M. and Noiman S. (2004) G Protein-Coupled Receptors: In Silico Drug Discovery in 3D. PNAS 101, 11304-11309.
Berman H. M., Westbrook J., Feng Z., Gilliland G., Bhat T. N., Weissig H., Shindyalov I. N. and Bourne P. E. (2000) The Protein Data Bank. Nucleic Acids Res. 28, 235-242.
Binkowski T. A., Naghibzadeh S. and Liang J. (2003) CASTp: Computed Atlas of Surface Topography of Proteins. Nucleic Acids Res. 31, 3352-3355.
Bohacek R. S., McMartin C. and Guida W. C. (1996) The Art and Practice of Structure-Based Drug Design: A Molecular Modeling Perspective. Med. Res. Rev. 16, 3-50.
Boot R. G., Blommaart E. F., Swart E., Ghauharali-van der Vlugt K., BO N., Moe C., Place A. and Aerts J. M. (2001) Identification of a Novel Acidic Mammalian Chitinase Distinct from Chitotriosidase. J. Biol. Chem. 276, 6770-6778.
Borghi C., Bacchelli S., Degli Esposti D. and Ambrosioni E. (2004) A Review of the Angiotensin-Converting Enzyme Inhibitor, Zofenopril, in the Treatment of Cardiovascular Diseases. Expert Opin. Pharmacother. 5, 1965-1977.
Bork P., Holm L. and Sander C. (1994) The Immunoglobulin Fold. Structural Classification, Sequence Patterns and Common Core. J. Mol. Biol. 242, 309-320.
Brunger A. T. (1992) Free R Value: A Novel Statistical Quantity for Assessing the Accuracy of Crystal Structures. Nature 355, 472-475.
Brunger A. T., Adams P. D., Clore G. M., DeLano W. L., Gros P., Grosse-Kunstleve R. W., Jiang J. S., Kuszewski J., Nilges M., Pannu N. S., Read R. J., Rice L. M., Simonson T. and Warren G. L. (1998) Crystallography & NMR System: A New Software Suite for Macromolecular Structure Determination. Acta Cryst. D54 (Pt 5), 905-921.
Burrell L. M., Johnston C. I., Tikellis C. and Cooper M. E. (2004) ACE2, A New Regulator of the Renin-Angiotensin System. Trends Endocrinol. Metab. 15, 166-169.
Cannon J. P., Haire R. N. and Litman G. W. (2002) Identification of Diversified Genes that Contain Immunoglobulin-Like Variable Regions in a Protochordate. Nat. Immunol. 3, 1200-1207.
Cannon J. P., Haire R. N., Schnitker N., Mueller M. G. and Litman G. W. (2004) Individual Protochordates Have Unique Immune-Type Receptor Repertoires. Curr. Biol. 14, R465-466.
Cantoni C., Ponassi M., Biassoni R., Conte R., Spallarossa A., Moretta A., Moretta L., Bolognesi M. and Bordo D. (2003) The Three-Dimensional Structure of the Human NK Cell Receptor NKp44, a Triggering Partner in Natural Cytotoxicity. Structure 11, 725-734.
Card G. L., Blasdel L., England B. P., Zhang C., Suzuki Y., Gillette S., Fong D., Ibrahim P. N., Ards D. R., Bollag G., Milburn M. V., Kim S. H., Schlessinger J. and Zhang K. Y. (2005) Family of Phosphodiesterase Inhibitors Discovered by Cocrystallography and Scaffold-based Drug Design. Nat. Biotechnol. 23, 201-207.
Carter P., Andersen C. A. and Rost B. (2003) DSSPcont: Continuous Secondary Structure Assignments for Proteins. Nucleic Acids Res. 31, 3293-3295.
Chen B., Piletsky S, and Turner A. P. (2002) High Molecular Recognition: Design of “Keys”. Comb. Chem. High Throughput Screen. 5, 409-427.
Chothia C., Gelfand I. and Kister A. (1998) Structural Determinants in the Sequences of Immunoglobulin Variable Domain. J. Mol. Biol. 278, 457-479.
Chothia C., Novotny J., Bruccoleri R. and Karplus M. (1985) Domain Association in Immunoglobulin Molecules. The Packing of Variable Domains. J. Mol. Biol. 186, 651-663.
Cleland W. W. (2000) Low-Barrier Hydrogen Bonds and Enzymatic Catalysis. Arch. Biochem. Biophys. 382, 1-5.
Clements C. S., Reid H. H., Beddoe T., Tynan F. E., Perugini M. A., Johns T. G., Bernard C. C. and Rossjohn J. (2003) The Crystal Structure of Myelin Oligodendrocyte Glycoprotein, a Key Autoantigen in Multiple Sclerosis. Proc. Natl. Acad. Sci. USA 100, 11059-11064.
Collaborative Computational Project N. (1994) The CCP4 Suite: Programs for Protein Crystallography. Acta Cryst. D50, 760-763.
Collins A. M., Sewell W. A. and Edwards M. R. (2003) Immunoglobulin gene rearrangement, repertoire diversity, and the allergic response. Pharmacol. Ther. 100, 157-170
Colman P. M. (1988) Structure of Antibody-Antigen Complexes: Implications for Immune Recognition. Adv. Immunol 43, 99-132.
Connolly M. L. (1983). Solvent-Accessible Surfaces of Proteins and Nucleic Acids. Science 221, 709-713.
Crackower M. A., Sarao R., Oudit G. Y., Yagil C., Kozieradzki I., Scanga S. E., Oliveira-dos-Santos A. J., da Costa J., Zhang L., Pei Y., Scholey J., Ferrario C. M., Manoukian A. S., Chappell M. C., Backx P. H., Yagil Y. and Penninger J. M. (2002) Angiotensin-Converting Enzyme 2 is an Essential Regulator of Heart Function. Nature 417, 822-828.
Dauter Z., Lamzin V. S. and Wilson K. S. (1997) The Benefits of Atomic Resolution. Curr. Opin. Struct. Biol. 7, 681-688.
Davis M. M. and Bjorkman P. J. (1988) T-Cell Antigen Receptor Genes and T-Cell Recognition. Nature 334, 395-402.
De Genst E., Handelberg F., Van Meirhaeghe A., Vynck S., Loris R., Wyns L. and Muyldermans S. (2004) Chemical Basis for the Affinity Maturation of a Camel Single Domain Antibody. J. Biol. Chem. 279, 53593-53601.
DeLano W. L. (2002) The PyMOL Molecular Graphics System. DeLano Scientific, San Carlos, Calif., USA.
Derewenda Z. S., Derewenda U. and Kobos P. M. (1994) (His)C Epsilon-H . . . O=C<Hydrogen Bond in the Active Sites of Serine Hydrolases. J. Mol. Biol. 241, 83-93.
Derewenda Z. S., Lee L. and Derewenda U. (1995) The Occurrence of C—H . . . O Hydrogen Bonds in Proteins. J. Mol. Biol. 252, 248-262.
Desiraju G. R. (1996) The CH—O Hydrogen Bond: Structural Implications and Supramolecular Design. Acc. Chem. Res. 29, 441-449.
Desmyter A., Transue T. R., Ghahroudi M. A., Thi M. H., Poortmans F., Hamers R., Muyldermans S. and Wyns L. (1996) Crystal Structure of a Camel Single-Domain VH Antibody Fragment in Complex with Lysozyme. Nat. Struct. Biol. 3, 803-811.
Dobson C. M. (2004) Chemical Space and Biology. Nat. Insight. Intro. pp. 2. Donoghue M., Hsieh F., Baronas E., Godabout K., Gosselin M., Stagliano N., Donovan M., Woolf B., Robison K., Jeyaseelan R., Breitbart R. E. and Acton S. (2000) A Novel Angiotensin-Converting Enzyme-Related Carboxypeptidase (ACE2) Converts Angiotensin Ito Angiotensin 1-9. Cir. Res. 87, E1-E9.
Dyson H. J. and Wright P. E. (2005) Intrinsically Unstructured Proteins and Their Functions. Nat. Rev. Mol. Cell. Biol. 6, 197-208.
Eason D. D., Cannon J. P., Haire R. N., Rast J. P., Ostrov D. A. and Litman G. W. (2004) Mechanisms of Antigen Receptor Evolution. Semin. Immunol. 16, 215-226.
Eisenmesser E. Z., Millet O., Labeikovsky W., Korzhnev D. M., Wolf-Watz M., Bosco D. A., Skalicky J. J., Kay L. E. and Kern D. (2005) Intrinsic Dynamics of an Enzyme Underlies Catalysis. Nature 438, 117-121.
Emsley P. and Cowtan K. (2004) Coot: Model-Building Tools for Molecular Graphics. Acta Cryst. D60, 2126-2132.
Evans S. V. (1993) SETOR: Hardware-Lighted Three-Dimensional Solid Model Representations of Macromolecules. J. Mol. Graphics. 11, 134-138, 127-128.
Ewing T. J., Makino S., Skillman A. G. and Kuntz I. D. (2001) DOCK 4.0: Search Strategies for Automated Molecular Docking of Flexible Molecule Databases. J. Comput. Aided Mol. Des. 15, 411-428.
Frauenfelder H., Parak F. and Young R. D. (1988) Conformational Substrates in Proteins. Ann. Rev. Biophys. Biophys. Chem. 17, 451-479.
Garcia K. C., Teyton L. and Wilson I. A. (1999) Structural Basis of T Cell Recognition. Ann. Rev. Immunol. 17, 369-397.
Garrett T. P., Wang J., Yan Y., Liu J. and Harrison S. C. (1993) Refinement and Analysis of the Structure of the First Two Domains of Human CD4. J. Mol. Biol. 234, 763-778.
Guex N., Diemand A. and Peitsch M. C. (1999) Protein Modelling for All. Trends Biochem. Sci. 24, 364-367.
Guex N. and Peitsch M. C. (1997) SWISS-MODEL and the Swiss-PdbViewer: An Environment for Comparative Protein Modeling. Electrophoresis 18, 2714-2723.
Hendrickson W. A., Horton J. R. and LeMaster D. M. (1990) Selenomethionyl Proteins Produced for Analysis by Multiwavelength Anomalous Diffraction (MAD): A Vehicle for Direct Determination of Three-Dimensional Structure. Embo Journal 9, 1665-1672.
Hernandez Prada J. A., Haire R. N., Cannon J. P., Litman G. W. and Ostrov D. A. (2004) Crystallization and Preliminary X-Ray Analysis of VCBP3 from Branchiostoma Floridae. Acta Cryst. D60, 2022-2024.
Hiom K., Melek M. and Gellert M. (1998) DNA Transposition by the RAG1 and RAG2 Proteins: A Possible Source of Oncogenic Translocations. Cell 94, 463-470.
Hogue C. W. (1997) Cn3D: A New Generation of Three-Dimensional Molecular Structure Viewer. Trends Biochem. Sci. 22, 314-316.
Holm L. and Sander C. (1999) Protein Folds and Families: Sequence and Structure Alignments. Nucleic Acids Res. 27, 244-247.
Huentelman M. J., Zubcevic J., Katovich M. J. and Raizada M. K. (2004) Cloning and Characterization of a Secreted Form of Angiotensin-Converting Enzyme 2. Regul. Pept. 122, 61-67.
Huggins M. L. (1943) The Structure of Fibrous Proteins. Chem. Rev. 32, 195-218.
Hunkapiller T. and Hood L. (1989) Diversity of the Immunoglobulin Gene Superfamily. Adv. Immunol. 44, 1-63.
Irwin J. J. and Shoichet B. K. (2005) ZINC—A Free Database of Commercially Available Compounds for Virtual Screening. J. Chem. Inf. Model. 45, 177-182.
Jaffe E. K. (2005) Morpheeins—A New Structural Paradigm for Allosteric Regulation. Trends Biochem. Sci. 30, 490-497.
Jancarik J. and Kim S. H. (1991) Sparse Matrix Sampling: A Screening Method for Crystallization of Proteins. J. Appl. Cryst. 24, 409-411.
Jones T. A., Zou J. Y., Cowan S. W. and Kjeldgaard (1991) Improved Methods for Building Protein Models in Electron Density Maps and the Location of Errors in these Models. Acta Cryst. A47 (Pt 2), 110-119.
Jung D. and Alt F. W. (2004) Unraveling V(D)J Recombination; Insights into Gene Regulation. Cell 116, 299-311.
Kang B. S., Devedjiev Y., Derewenda U. and Derewenda Z. S. (2004) The PDZ2 Domain of Syntenin at Ultra-High Resolution: Bridging the Gap between Macromolecular and Small Molecule Crystallography. J. Mol. Biol. 338, 483-493.
Kantardjieff K. A. and Rupp B. (2003) Matthews Coefficient Probabilities: Improved Estimates for Unit Cell Contents of Proteins, DNA, and Protein-Nucleic Acid Complex Crystals. Protein Sci. 12, 1865-1871.
Katovich M. J., Grobe J. L., Huentelman M. and Raizada M. K. (2005) Angiotensin-Converting Enzyme 2 as a Novel Target for Gene Therapy for Hypertension. Exp. Physiol. 90, 299-305.
Kawabata S., Nagayama R., Hirata M., Shigenaga T., Agarwala K. L., Saito T., Cho J., Nakajima H., Takagi T. and Iwanaga S. (1996) Tachycitin, a Small Granular Component in Horseshoe Crab Hemocytes, is an Antimicrobial Protein with Chitin-Binding Activity. J. Biochem. 120, 1253-1260.
Kitchen D. B., Decomez H., Fun J. R. and Bajorath J. (2004) Docking and Scoring in Virtual Screening for Drug Discovery: Methods and Applications. Nat. Rev. Drug Discov. 3, 935-949.
Kolodny R., Koehl P. and Levitt M. (2005) Comprehensive Evaluation of Protein Structure Alignment Methods: Scoring by Geometric Measures. J. Mol. Biol. 346, 1173-1188.
Kuntz I. D., Blaney J. M., Oatley S. J., Langridge R. and Ferrin T. E. (1982) A Geometric Approach to Macromolecule-Ligand Interactions. J. Mol. Biol. 161, 269-288.
Laird D. J., De Tomaso A. W., Cooper M. D. and Weissman I. L. (2000) 50 Million Years of Chordate Evolution: Seeking the Origins of Adaptive Immunity. Proc. Natl. Acad. Sci. USA 97, 6924-6926.
Langer T. and Hoffmann R. D. (2001) Virtual Screening: An Effective Tool for Lead Structure Discovery? Curr. Pharm. Des. 7, 509-527.
Laskowski R. A., Moss D. S. and Thornton J. M. (1993) Main-Chain Bond Lengths and Bond Angles in Protein Structures. J. Mol. Biol. 231, 1049-1067.
Leahy D. J., Axel R. and Hendrickson W. A. (1992) Crystal Structure of A Soluble Form of the Human T Cell Coreceptor CD8 at 2.6 A Resolution. Cell 68, 1145-1162.
Lefranc M. P., Giudicelli V., Ginestoux C., Bosc N., Folch G., Guiraudou D., Jabado-Michaloud J., Magris S., Scaviner D., Thouvenin V., Combres K., Girod D., Jeanjean S., Protat C., Yousfi-Monod M., Duprat E., Kaas Q., Pommie C., Chaume D. and Lefranc G. (2004) IMGT-ONTOLOGY for Immunogenetics and Immunoinformatics. In Silico Biol. 4, 17-29.
Li H., Lebedeva M. I., Llera A. S., Fields B. A., Brenner M. B. and Mariuzza R. A. (1998) Structure of the Vdelta Domain of a Human Gammadelta T-Cell Antigen Receptor. Nature 391, 502-506.
Lipinski C. A., Lombardo F., Dominy B. W. and Feeney P. J. (1997) Experimental and Computational Approaches to Estimate Solubility and Permeability in Drug Discovery and Development Settings. Adv. Drug Delivery Rev. 23(1-3), 3-25.
Litman G. W., Anderson M. K. and Rast J. P. (1999) Evolution of Antigen Binding Receptors. Ann. Rev. Immunol. 17, 109-147.
Litman G. W., Cannon J. P. and Dishaw L. J. (2005) Reconstructing Immune Phylogeny: New Perspectives. Nat. Rev. Immunol. 5, 866-879.
Loll B., Raszewski G., Saenger W. and Biesiadka J. (2003) Functional Role of C(Alpha)-H . . . O Hydrogen Bonds between Transmembrane Alpha-Helices in Photosystem I. J. Mol. Biol. 328, 737-747.
Luz J. G., Huang M., Garcia K. C., Rudolph M. G., Apostolopoulos V., Teyton L. and Wilson I. A. (2002) Structural Comparison of Allogeneic and Syngeneic T Cell Receptor-Peptide-Major Histocompatibility Complex Complexes: A Buried Alloreactive Mutation Subtly Alters Peptide Presentation Substantially Increasing V(Beta) Interactions. J. Exp. Med. 195, 1175-1186.
Marchler-Bauer A., Anderson J. B., Cherukuri P. F., DeWeese-Scott C., Geer L. Y., Gwadz M., He S., Hurwitz D. I., Jackson J. D., Ke Z., Lanczycki C. J., Liebert C. A., Liu C., Lu F., Marchler G. H., Mullokandov M., Shoemaker B. A., Simonyan V., Song J. S., Thiessen P. A., Yamashita R. A., Yin J. J., Zhang D. and Bryant S. H. (2005) CDD: A Conserved Domain Database for Protein Classification. Nucleic Acids Res. D33, 192-196.
Matthews B. W. (1968) Solvent Content of Protein Crystals. J Mol. Biol. 33, 491-497.
Matthews B. W. (1976) X-Ray Crystallographic Studies of Proteins. Ann. Rev. Phys. Chem. 27, 493-523.
McCammon J. A. (2005) Target Flexibility in Molecular Recognition. Biochim. Biophys. Acta. 1754, 221-4.
McDonald I. K. and Thornton J. M. (1994) Satisfying Hydrogen Bonding Potential in Proteins. J. Mol. Biol. 238, 777-793.
McPherson A. (1999) Crystallization of Biological Macromolecules (Cold Spring Harbor Laboratory, 1st edition) pp. 586.
McRee D. E. (1999) XtalView/Xfit—A Versatile Program for Manipulating Atomic Coordinates and Electron Density. J. Struct. Biology 125, 156-165.
Medzhitov R. and Janeway C. Jr. (2000) Innate Immunity. N. Engl. J. Med. 343, 338-344.
Meibom K. L., Blokesch M., Dolganov N. A., Wu C. Y. and Schoolnik G. K. (2005) Chitin Induces Natural Competence in Vibrio Cholerae. Science 310, 1824-1827.
Meng E. C., Gschwend D. A., Blaney J. M. and Kuntz I. D. (1993) Orientational Sampling and Rigid-Body Minimization in Molecular Docking. Proteins 17, 266-278.
Meng E. C., Shoichet K. and Kuntz I. D. (1992) Automated Docking with Grid Based Energy Evaluation. J. Comp. Chem. 13, 505-524.
Merritt E. A. (1994) Raster3D Version 2.0. A Program for Photorealistic Molecular Graphics. Acta Cryst. D50, 869-873.
Merritt E. A. (1999) Comparing Anisotropic Displacement Parameters in Protein Structures. Acta Cryst. D55 (Pt 12), 1997-2004.
Miura K., Daviglus M. L., Greenland P. and Stamler J. (2001) Making Prevention and Management of Hypertension Work. J. Hum. Hypertens. 15, 1-3.
Monod M. Y., Giudicelli V., Chaume D. and Lefranc M. P. (2004) IMGT/JunctionAnalysis: The First Tool for the Analysis of the Immunoglobulin and T Cell Receptor Complex. Bioinformatics 20 Suppl 1, 1379-1385.
Myers G. and Durbin R. (2003) A Table-Driven, Full-Sensitivity Similarity Search Algorithm. J. Comput. Biol. 10, 103-117.
Natesh R., Schwager S. L., Sturrock E. D. and Acharya K. R. (2003) Crystal Structure of the Human Angiotensin-Converting Enzyme-Lisinopril Complex. Nature 421, 551-554.
Nicholls A., Sharp K. and Honig B. (1991) GRASP: A Molecular Surface Graphics Program. PROTEINS, Structure, Function and Genetics. 11, 281.
Orengo C. A. and Thornton J. M. (2005) Protein Families and their Evolution-Astructural Perspective. Annu. Rev. Biochem. 74, 867-900.
Ostrov D. A., Shi W., Schwartz J. C., Almo S. C. and Nathenson S. G. (2000) Structure of Murine CTLA-4 and its Role in Modulating T Cell Responsiveness. Science 290, 816-819.
Otwinowski Z., Borek D., Majewski W. and Minor W. (2003) Multiparametric Scaling of Diffraction Intensities. Acta Crystallographica. Section a, Crystal Physics, Diffraction, Theoretical and General Crystallography 59, 228-234.
Otwinowski Z. and Minor W. (1997) Processing of X-ray Diffraction Data Collected in Oscillation Mode. Methods Enzymol. 276, 307-326.
Peitsch M. C. (1996) ProMod and Swiss-Model: Internet-Based Tools for Automated Comparative Protein MOdelling. Biochem. Soc. Trans. 24, 274-279.
Peitsch M. C., Schwede T. and Guex N. (2000) Automated Protein Modelling—the Proteome in 3D. Pharmacogenomics 1, 257-266.
Potapov V., Sobolev V., Edelman M., Kister A. and Gelfand 1. (2004) Protein-1-Protein Recognition: Juxtaposition of Domain and Interface Cores in Immunoglobulins and Other Sandwich-Like Proteins. J. Mol. Biol. 342, 665-679.
Rast J. P., Anderson M. K., Strong S. J., Litman R. T. and Litman G. W. (1997) a, t3, y, and S T Cell Antigen Receptor Genes Arose Early in Vertebrate Phylogeny. Immunity 6, 1-11.
Rast J. P. and Litman G. W. (1998) Towards Understanding the Evolutionary Origins and Early Diversification of Rearranging Antigen Receptors. Immunological Reviews 166, 79-86.
Rayment I., Rypniewski W. R., Schmidt-Base K., Smith R., Tomchick D. R., Benning M. M., Winkelmann D. A., Wesenberg G. and Holden H. M. (1993) Three-Dimensional Structure of Myosin Subfragment 1: A Molecular motor. Science. 261, 50-58.
Renkema G. H., Boot R. G., Muijsers A. O., Donker-Koopman W. E. and Aerts J. M. (1995) Purification and Characterization of Human Chitotriosidase, a Novel Member of the Chitinase Family of Proteins. J. Biol. Chem. 270, 2198-2202.
Renkema G. H., Boot R. G., Strijland A., Donker-Koopman W. E., van den Berg W., Muijsers A. O. and Aerts J. M. (1997) Synthesis, Sorting and Processing into Distinct Isoforms of Human Macrophage Chitotriosidase. Eur. J. Biochem. 244, 279-285.
Richards A. G. and Richards P. A. (1977) The Peritrophic Membranes of Insects. Annu. Rev. Entomol. 22, 219-240.
Rini J. M., Schulze-Gahmen U. and Wilson I. A. (1992) Structural Evidence for Induced Fit as a Mechanism for Antibody-Antigen Recognition. Science 255, 959-965.
Ross E. D., Minton A. and Wickner R. B. (2005) Prion domains: sequences, structures and interactions. Nat. Cell Biol. 7, 1039-1044.
Rudolph M. G., Luz J. G. and Wilson I. A. (2002) Structural and Thermodynamic Correlates of T Cell Signaling. Annu. Rev. Biophys. Biomol. Struct. 31, 121-149.
Rudolph M. G. and Wilson I. A. (2002) The Specificity of TCR/pMHC Interaction. Curr. Opin. Immunol. 14, 52-65.
Ruiz-Ortega M., Lorenzo O., Ruperez M., Estevan V., Suzuki Y., Mezzano S., Plaza J. J. and Egido J. (2001) Role of the Renin-Angiotensin System in Vascular Diseases: Expanding the Field. Hypertension. 38, 1382-1387.
Rypniewski W. R., Holden H. M. and Rayment I. (1993) Structural Consequences of Reductive Methylation of Lysine Residues in Hen Egg White Lysozyme: an X-ray Analysis at 1.8-A Resolution. Biochemistry 32, 9851-9858.
Schneider T. R. and Sheldrick G. M. (2002) Substructure Solution with SHELXD. Acta Cryst. D58, 1772-1779.
Schwede T., Kopp J., Guex N. and Peitsch M. C. (2003) SWISS-MODEL: An Automated Protein Homology-Modeling Server. Nucleic Acids Res. (Online) 31, 3381-3385.
Sheldrick G. M. and Schneider T. R. (1997) SHELXL: High-Resolution Refinement. Methods Enzymol. 277, 319-343.
Shen Z. and Jacobs-Lorena M. (1999) Evolution of Chitin-Binding Proteins in Invertebrates. J. Mol. Evol. 48, 341-347.
Simonds W. F. (1999) G Protein Regulation of Adenylate Cyclase. Trends Pharmacol. Sci. 20, 66-73.
Stanfield R. L., Dooley H., Flajnik M. F. and Wilson I. A. (2004) Crystal Structure of a Shark Single-Domain Antibody V Region in Complex with Lysozyme. Science 305, 1770-1773.
Stanfield R. L., Takimoto-Kamimura M., Rini J. M., Profy A. T. and Wilson I. A. (1993) Major Antigen-Induced Domain Rearrangements in an Antibody: Structure 1, 83-93.
Streltsov V. A., Varghese J. N., Carmichael J. A., Irving R. A., Hudson P. J. and Nuttall S. D. (2004) Structural Evidence for Evolution of Shark Ig New Antigen Receptor Variable Domain Antibodies from a Cell-Surface Receptor. Proc. Natl. Acad. Sci. USA 101, 12444-12449.
Strong S. J., Mueller M. G., Litman R. T., Hawke N. A., Haire R. N., Miracle A. L., Rast J. P., Amemiya C. T. and Litman G. W. (1999) A Novel Multigene Family Encodes Diversified Variable Regions. Proc. Natl. Acad. Sci. USA 96, 15080-15085.
Suetake T., Aizawa T., Koganesawa N., Osaki T., Kobashigawa Y., Demura M., Kawabata S., Kawano K., Tsuda S, and Nitta K. (2002) Production and Characterization of Recombinant Tachycitin, the Cys-Rich Chitin-Binding Protein. Protein Eng. 15, 763-769.
Suetake T., Tsuda S., Kawabata S., Miura K., Iwanaga S., Hikichi K., Nitta K. and Kawano K. (2000) Chitin-Binding Proteins in Invertebrates and Plants Comprise a Common Chitin-Binding Structural Motif. J. Biol. Chem. 275, 17929-17932.
Suri M. F. K., Kirmani J. F. and Divani A. A. AHA News Release Sep. 3, 2004, at www.americanheart.org.
Taylor R. and O. Kennard (1982) Crystallographic Evidence for the Existence of CH—O, CH—N, and CH—Cl Hydrogen Bonds. J. Am. Chem. Soc. 104, 5063-5070.
Terwilliger T. C. (2000) Maximum-Likelihood Density Modification. Acta Cryst. D56, 965-972.
Terwilliger T. C. (2002) Automated Main-Chain Model Building by Template Matching and Iterative Fragment Extension. Acta Cryst. D59, 38-44.
Terwilliger T. C. and Berendzen J. (1999) Automated MAD and MIR Structure Solution. Acta Cryst. D55 (Pt 4), 849-861.
Tipnis S. R., Hooper N. M., Hyde R., Karran E., Christie E. and Turner A. J. (2000) A Human Homolog of Angiotensin-Converting Enzyme. Cloning and Functional Expression as a Captopril-Insensitive Carboxypeptidase. J. Biol. Chem. 275, 33238-33243.
Tjoelker L. W., Gosting L., Frey S., Hunter C. L., Trong H. L., Steiner B.; Brammer H. and Gray P. W. (2000) Structural and Functional Definition of the Human Chitinase Chitin-Binding Domain. J. Biol. Chem. 275, 514-520.
Tonegawa S. (1983) Somatic Generation of Antibody Diversity. Nature 302, 575-581.
Towler P., Staker B., Prasad S. G., Menon S., Tang J., Parsons T., Ryan D., Fisher M., Williams D., Dales N. A., Patane M. A. and Pantoliano M. W. (2004) ACE2 X-Ray Structures Reveal a Large Hinge-Bending Motion Important for Inhibitor Binding and Catalysis. J. Biol. Chem. 279, 17996-18007.
Unger T. (2003) Blood Pressure Lowering and Renin-Angiotensin System Blockade. J. Hypertens. 21 suppl 6, S3-S7.
van den Berg T. K., Yoder J. A. and Litman G. W. (2004) On the Origins of Adaptive Immunity Innate Immune Receptors Join the Tale. Trends in Immunology 25, 11-16.
van Eijk M., van Roomen C. P., Renkema G. H., Bussink A. P., Andrews L., Blommaart E. F., Sugar A., Verhoeven A. J., Boot R. G. and Aerts J. M. (2005)
Characterization of Human Phagocyte-Derived Chitotriosidase, a Component of Innate Immunity. Int. Immunol. 17, 1505-1512.
van Gunsteren W. F. (1996) Biomolecular Simulation: The GROMOS96 Manual and User Guide. 4-31.
van Raaij M. J., Chouin E., van der Zandt H., Bergelson J. M. and Cusack S. (2000) Dimeric Structure of the Coxsackievirus and Adenovirus Receptor D1 Domain at 1.7 A Resolution. Structure Fold Des. 8, 1147-1155.
Vickers C., Hales P., Kaushik V., Dick L., Gavin J., Tang J., Godbout K., Parsons T., Baronas E., Hsieh F., Acton S., Patane M., Nichols A. and Tummino P. (2002) Hydrolysis of Biological Peptides by Human Angiotensin-Converting Enzyme-Related Carboxypeptidase. J. Biol. Chem. 277, 14838-14843.
Volkman B. F., Lipson D., Wemmer D. E. and Kern D. (2001) Two-State Allosteric Behavior in a Single-Domain Signaling Protein. Science 291, 2429-2433.
Wahl M. C., Rao S. T. and Sundaralingam M. (1996) The Structure of R(UUCGCG) Has a 5′-UU-Overhang Exhibiting Hoogsteen-Like Trans U.0 Base Pairs. Nat. Struct. Biol. 3, 24-31.
Wahl M. C. and Sundaralingam M. (1997) C—H . . . O Hydrogen Bonding in Biology. Trends Biochem. Sci. 22, 97-102.
Wallace A. C., Laskowski R. A. and Thornton J. M. (1995) LIGPLOT: A Program to Generate Schematic Diagrams of Protein-Ligand Interactions. Protein Eng. 8, 127-134.
Walters W. P., Stahl M. T. and Murcko M. A. (1998) Virtual Screening—An Overview. Drug Discov. Today 3, 160-178.
Wang J. W., Chen J. R., Gu Y. X., Zheng C. D., Jiang F., Fan H. F., Terwilliger T. C. and Hao Q. (2004) SAD Phasing by Combination of Direct Methods with the SOLVE/RESOLVE Procedure. Acta Cryst. D60, 1244-1253.
Watson F. L., Puttmann-Holgado R., Thomas F., Lamar D. L., Hughes M., Kondo M., Rebel V. I. and Schmucker D. (2005) Extensive Diversity of Ig-Superfamily Proteins in the Immune System of Insects. Science 309, 1874-1878.
Weeks C. M., Adams P. D., Berendzen J., Brunger A. T., Dodson E. J., Grosse-Kunstleve R. W., Schneider T. R., Sheldrick G. M., Terwillinger T. C., Turkenburg M. G. and Uson I. (2003) Automatic Solution of Heavy-Atom Substructures. Substructure Solution with SHELXD. Methods Enzymol. 374, 37-83.
Wolf-Watz M., That V., Henzler-Wildman K., Hadjipavlou G., Eisenmesser E. Z. and Kern D. (2004) Linkage between dynamics and catalysis in a thermophilic-mesophilic enzyme pair. Nature Struct. & Mol. Biol. 11, 945-949.
Yoder J. A., Litman R. T., Mueller M. G., Desai S., Dobrinski K. P., Montgomery J. S., Buzzeo M. P., Ota T., Amemiya C. T., Trede N. S., Wei S., Djeu J. Y., Humphray S., Jekosch K., Hernandez Prada J. A., Ostrov D. A. and Litman G. W. (2004) Resolution of the Novel Immune-Type Receptor Gene Cluster in Zebrafish. Proc. Natl. Acad. Sci. USA 101, 15706-15711.
Zaccai N. R., Maenaka K., Maenaka T., Crocker P. R., Brossmer R., Kelm S. and Jones E. Y. (2003) Structure-Guided Design of Sialic Acid-Based Siglec Inhibitors and Crystallographic Analysis in Complex with Sialoadhesin. Structure (Camb) 11, 557-567.
Zaman M. A., Oparil. S. and Calhoun D. A. (2002) Drugs Targeting the Renin-Angiotensin-Aldosterone System. Nat. Rev. Drug Discov. 1, 621-636.
Zemlin M., Schelonka R. L., Bauer K. and Schroeder H. W. Jr. (2002) Regulation and Chance in the Ontogeny of B and T Cell Antigen Receptor Repertoires. Immunol. Res. 26, 265-278
Zhang J. and Madden T. L. (1997) PowerBLAST: A New Network BLAST Application for Interactive or Automated Sequence Analysis and Annotation. Genome Res. 7, 649-656.
Zhang S. M., Adema C. M., Kepler T. B. and Loker E. S. (2004) Diversification of Ig Superfamily Genes in an Invertebrate. Science 305, 251-254.
Zhurkin V. B., Raghunathan G., Ulyanov N. B., Camerini-Otero R. D. and Jernigan R. L. (1994) A Parallel DNA Triplex as a Model for the Intermediate in Homologous Recombination. J. Mol. Biol. 239, 181-200.

The disclosures of each and every patent, patent application and publication cited herein are hereby incorporated herein by reference in their entirety.

Although the invention has been disclosed with reference to specific embodiments, it is apparent that other embodiments and variations of the invention may be devised by others skilled in the art without departing from the true spirit and scope of the invention. The sentences are intended to be construed to include all such embodiments and equivalent variations.

1. A method of treating a subject suffering from or susceptible to cardiovascular disease or hypertension, the method comprising administering to a subject in need thereof a therapeutically effective amount of an angiotensin converting enzyme 2 (ACE2) activator, to thereby treat the subject suffering from or susceptible to cardiovascular disease or hypertension.

2. (canceled)

3. The method of claim 1, wherein the ACE activator is represented by Formula (I):
Ar—(Y)ₙ (I)
wherein,
Ar is a polycyclic fused aromatic moiety;
Y represents a hydrogen bond donor or acceptor; and
n is an integer from 2 to 8; or a pharmaceutically acceptable salt or prodrug thereof.

4. The method of claim 1, wherein the ACE activator is represented by Formula (II):
Second Preliminary Amendment 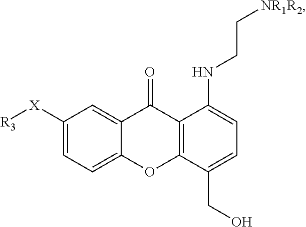
in which
X is O or S;
R₁ and R₂ are independently hydrogen, optionally substituted C₁-C₈alkyl, optionally substituted C₃-C₈cycloalkyl, optionally substituted C₂-C₈ alkenyl, optionally substituted C₂-C₈alkynyl, optionally substituted C₁-C₈alkanoyl, or optionally substituted aryl; and
R₃ is optionally substituted C₁-C₈alkyl, optionally substituted C₃-C₈cycloalkyl, optionally substituted C₂-C₈ alkenyl, optionally substituted C₂-C₈alkynyl, optionally substituted C₁-C₈alkanoyl, optionally substituted C₁-C₈alkanoyl or optionally substituted C₁-C₈alkylsulfonyl, optionally substituted C₁-C₈arylsulfonyl, or optionally substituted aryl;
or a pharmaceutically acceptable salt or prodrug thereof.

5. The method of claim 1, wherein the ACE activator is selected from 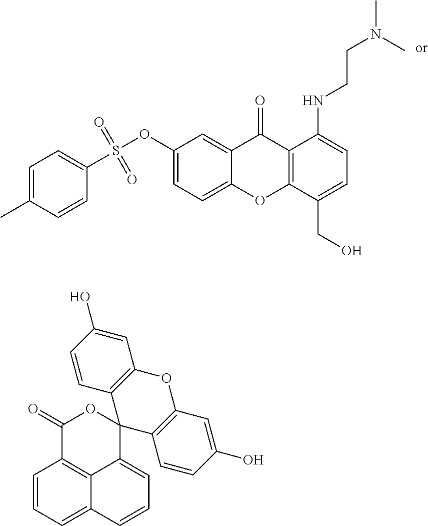
or a pharmaceutically acceptable salt or prodrug thereof.

6. A method for identifying a compound that activates ACE2, the method comprising:
a) obtaining a crystal structure of ACE2 or obtaining information relating to the crystal structure of ACE2, and
b) modeling a test compound into or on the crystal structure coordinates to determine whether the compound activates ACE2.

7. The method of claim 6, wherein the step of modeling comprises modeling or determining the ability of the compound to bind to or associate with a binding pocket defined by structure coordinates of one or more ACE2 amino acid residues Lys94, Tyr196, Gly205 and His195, or a binding pocket defined by structure coordinates of one or more ACE2 amino acid residues Gln98, Gln101 and Gly205.

8. (canceled)

9. A method for identifying a compound that modulates the activity of ACE2, the method comprising using the atomic coordinates of one or more ACE2 amino acid residues selected from Lys94, Tyr196, Gly205 and His195 or one or more ACE2 amino acid residues selected from Gln98, Gln101 and Gly205, to generate a three-dimensional structure of a molecule comprising an ACE2 binding pocket, and employing the three-dimensional structure to identify a compound that modulates the activity of ACE2.

10.-15. (canceled)

16. A pharmaceutical composition comprising a compound of Table 1, or a pharmaceutically acceptable salt or prodrug thereof, together with a pharmaceutically acceptable carrier.

17. A method of treating a subject suffering from or susceptible to acute lung injury, cardiac or renal fibrosis, or pulmonary hypertension, the method comprising administering to the subject an effective amount of an ACE2 activator compound or a compound capable of activating ACE2 activity or expression in a cell, such that the subject is treated.

18.-22. (canceled)

23. The method of claim 17, wherein the compound is represented by Formula (I):
Ar—(Y)ₙ (I)
wherein,
Ar is a polycyclic fused aromatic moiety;
Y represents a hydrogen bond donor or acceptor; and
n is an integer from 2 to 8; or a pharmaceutically acceptable salt or prodrug thereof.

24. The method of claim 17, wherein the compound is represented by Formula (II): 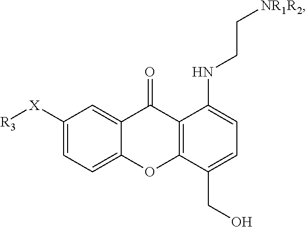
in which
X is O or S;
R₁ and R₂ are independently hydrogen, optionally substituted C₁-C₈alkyl, optionally substituted C₃-C₈cycloalkyl, optionally substituted C₂-C₈ alkenyl, optionally substituted C₂-C₈alkynyl, optionally substituted C₁-C₈alkanoyl, or optionally substituted aryl; and
R₃ is optionally substituted C₁-C₈alkyl, optionally substituted C₃-C₈cycloalkyl, optionally substituted C₂-C₈ alkenyl, optionally substituted C₂-C₈alkynyl, optionally substituted C₁-C₈alkanoyl, optionally substituted C₁-C₈alkanoyl or optionally substituted C₁-C₈alkylsulfonyl, optionally substituted C₁-C₈arylsulfonyl, or optionally substituted aryl;
or a pharmaceutically acceptable salt or prodrug thereof.

25. The method of claim 17, wherein the compound is selected from 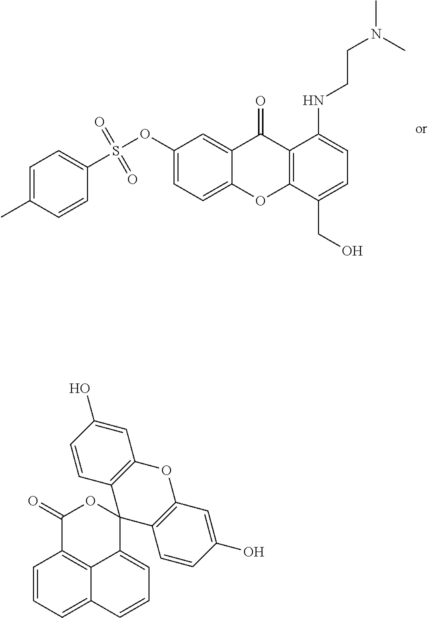
or a pharmaceutically acceptable salt or prodrug thereof.

26. (canceled)

27. A pharmaceutical composition comprising a compound represented by Formula (II): 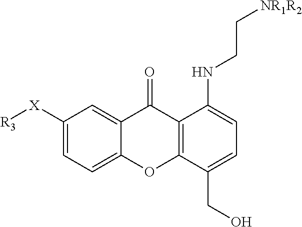
in which
X is O or S.
R₁ and R₂ are independently hydrogen, optionally substituted C₁-C₈alkyl, optionally substituted C₃-C₈cycloalkyl, optionally substituted C₂-C₈ alkenyl, optionally substituted C₂-C₈alkynyl, optionally substituted C₁-C₈alkanoyl or optionally substituted aryl; and
R₃ is substitute C₁-C₈alkyl, optionally substituted C₃-C₈cycloalkyl, optionally substituted C₂-C₈ alkenyl, optionally substituted C₂-C₈alkynyl, optionally substituted C₁-C₈alkanoyl optionally substituted C₁-C₈alkanoyl or optionally substituted C₁-C₈alkylsulfonyl, optionally substituted C₁-C₈arylsulfonyl, or optionally substituted aryl;
or a pharmaceutically acceptable salt or prodrug thereof and a pharmaceutically acceptable carrier.
